(19) United States
(12) Patent Application Publication
(10) Pub. No.: US 2018/0103928 A1
(43) Pub. Date: Apr. 19, 2018

(54) ADJUSTABLE HEADSET

(71) Applicant: Neural Analytics, Inc., Los Angeles, CA (US)

(72) Inventors: Michael Costa, Los Angeles, CA (US); Trevor Dunlop, Los Angeles, CA (US); Roman Flores, II, Los Angeles, CA (US); Robert Hamilton, Los Angeles, CA (US); Matthew Hutter, Los Angeles, CA (US); Michael O'Brien, Los Angeles, CA (US); Leo Petrossian, Los Angeles, CA (US); Shankar Radhakrishnan, Los Angeles, CA (US); Lane Stith, Los Angeles, CA (US); Seth J. Wilk, Los Angeles, CA (US); Jan Zwierstra, Los Angeles, CA (US)

(21) Appl. No.: 15/785,384

(22) Filed: Oct. 16, 2017

Related U.S. Application Data
(60) Provisional application No. 62/409,060, filed on Oct. 17, 2016.
(60) Provisional application No. 62/412,755, filed on Oct. 25, 2016.

(51) Int. Cl. A61B 8/00; A61B 8/08

(52) U.S. Cl. CPC A61B 8/4209; A61B 8/488; A61B 3/0075; A61B 2562/12; A61B 8/4227

(57) ABSTRACT

A headset for placing on a head of a subject is provided. The headset includes a device including a probe. The headset further includes at least one adjustable mechanism configured to adjust a fit of the headset such that the device is aligned with an acoustic window of the head of the subject.

CROSS-REFERENCE TO RELATED PATENT APPLICATIONS

This application claims the benefit of and priority to U.S. provisional patent application Ser. No. 62/409,060, filed Oct. 17, 2016, the contents of which are incorporated herein by reference in its entirety. This application claims the benefit of and priority to U.S. provisional patent application Ser. No. 62/412,755, filed Oct. 25, 2016, the contents of which are incorporated herein by reference in its entirety.

### BACKGROUND

For devices utilizing a headset whose performance is optimized by remaining stable with respect to a user's head (e.g., optical devices, surgical devices, scanning devices, medical diagnostic devices, automated Transcranial Doppler devices, and so on), alignment and movement of the device during operation can be a concern. For example, if the headset connected to the device is disturbed by muscle movements of the headset user (e.g., by muscles located on the head), the device may become misaligned, resulting in false or inadequate readings or performance by the device. In addition, the headset can be cumbersome to initially place on the head, to setup, and to use due to the nature of flexible straps and lack of rigidity holding the headset together. For example, adjustment of a side, a front, or a back of a headset (e.g., for fit) may affect the overall general symmetry of the headset, which may result in unwanted movements at other locations of the headset, and thus making it difficult to align and stabilize the device attached to the headset at an optimal position.

### SUMMARY

According to various embodiments, there is provided a headset that allows a device attached thereto to remain fixed at an optimal position with respect to a user's head during operation, without being disturbed by muscular movements by the user wearing the headset. In addition, according to various embodiments, there is provided a headset that is easy to adjust and setup, and that provides sufficient rigid support to position and hold the device attached to the headset in a desired location during adjustment of the headset or device.

According to various embodiments, there is provided a headset for placing on a head of a subject. The headset includes a device including a probe. The headset further includes at least one adjustable mechanism configured to adjust a fit of the headset such that the device is aligned with an acoustic window of the head of the subject.

In some embodiments, the at least one adjustable mechanism includes a temple adjustment mechanism configured to telescope to elongate or shorten a length of the headset at a temple of the head of the subject.

In some embodiments, the at least one adjustable mechanism further includes a tip adjustment mechanism configured to telescope to elongate or shorten the length of the headset at an ear of the head of the subject.

In some embodiments, the device is interposed between the temple adjustment mechanism and the tip adjustment mechanism along the length of the headset.

In some embodiments, the at least one adjustable mechanism includes an adjustable pad located between the headset and the head of the subject and is configured to contact the head of the subject.

In some embodiments, the adjustable pad has a first thickness and is replaceable with a replacement adjustable pad having a second thickness different from the first thickness.

In some embodiments, the headset further includes a head strap configured to span over a top of the head of the subject.

In some embodiments, the device further includes a robotic mechanism attached to the probe for automatic control of the probe.

In some embodiments, the probe includes an ultrasound probe.

In some embodiments, the acoustic window includes a temporal acoustic window of the head of the subject.

According to various embodiments, there is provided a method of manufacturing a headset for placing on a head of a subject. The method includes providing a device including a probe. The method further includes providing at least one adjustable mechanism configured to adjust a fit of the headset such that the device is aligned with an acoustic window of the head of the subject.

In some embodiments, the at least one adjustable mechanism includes a temple adjustment mechanism configured to telescope to elongate or shorten a length of the headset at a temple of the head of the subject.

In some embodiments, the at least one adjustable mechanism further includes a tip adjustment mechanism configured to telescope to elongate or shorten the length of the headset at an ear of the head of the subject.

In some embodiments, the device is interposed between the temple adjustment mechanism and the tip adjustment mechanism along the length of the headset.

In some embodiments, the at least one adjustable mechanism includes an adjustable pad located between the headset and the head of the subject and is configured to contact the head of the subject.

In some embodiments, the adjustable pad has a first thickness and is replaceable with a replacement adjustable pad having a second thickness different from the first thickness.

In some embodiments, the method further includes providing a head strap configured to span over a top of the head of the subject.

In some embodiments, the device further includes a robotic mechanism attached to the probe for automatic control of the probe.

In some embodiments, the probe includes an ultrasound probe.

In some embodiments, the acoustic window includes a temporal acoustic window of the head of the subject.

### BRIEF DESCRIPTION OF THE FIGURES

Features, aspects, and advantages of the present invention will become apparent from the following description and the accompanying example embodiments shown in the drawings, which are briefly described below.

FIG. 1A illustrates a side view of a diagram illustrating the skeletal anatomy of a human head.

FIG. 1B illustrates a side view of a diagram illustrating the muscular anatomy of a human head.

FIG. 2 illustrates a side view of an adjustable headset configured for telescoping adjustment according to various embodiments.

FIG. 3 illustrates a side view of an adjustable headset including a temple tip and a foot pad according to various embodiments.

FIG. 4A illustrates a side view of an adjustable headset including a telescoping angled headband and a head strap according to various embodiments.

FIG. 4B illustrates a side view of the adjustable headset shown in FIG. 4A without the head strap according to various embodiments.

FIG. 4C illustrates a side view of the adjustable headset shown in FIG. 4B including an adjustable pad according to various embodiments.

FIG. 5 illustrates a perspective view of an adjustable headset including a spring arm overhead strap according to various embodiments.

FIG. 6 illustrates a perspective view of an adjustable headset including a spring arm overhead strap and an adjustable head support strap according to various embodiments.

FIG. 7 illustrates a perspective view of an adjustable headset including a spring arm overhead strap and a telescoping probe adjustment support according to various embodiments.

FIG. 8 illustrates a top perspective view of an adjustable headset including a spring arm overhead strap and a screw-tightened telescoping probe adjustment support according to various embodiments.

FIG. 9 illustrates a perspective view of an adjustable headset including a spring-loaded foldable arm according to various embodiments.

FIG. 10A illustrates a perspective view of an adjustable headset including a foldable arm according to various embodiments.

FIG. 10B illustrates a side view of the adjustable headset shown in FIG. 10A according to various embodiments.

FIG. 11A illustrates a perspective view of an adjustable headset including an adjustable side arm according to various embodiments.

FIG. 11B illustrates a top perspective view of the adjustable headset shown in FIG. 11A according to various embodiments.

FIG. 11C illustrates a front view of the adjustable headset shown in FIG. 11A according to various embodiments.

FIG. 12 illustrates a side view of an adjustable headset configured to provide adjustment along a radius according to various embodiments.

FIG. 13 illustrates a front view of an adjustable headset including a spring hinge clamp according to various embodiments.

FIG. 14 illustrates a perspective view of an adjustable headset including a spring hinge clamp according to various embodiments.

FIG. 15A illustrates a perspective view of an adjustable headset having two separated sections according to various embodiments.

FIG. 15B illustrates a side view of the adjustable headset shown in FIG. 15A according to various embodiments.

FIG. 15C illustrates a top view of the adjustable headset shown in FIG. 15A according to various embodiments.

FIG. 16A illustrates a perspective view of an adjustable headset including a handle grip according to various embodiments.

FIG. 16B illustrates a side view of the adjustable headset shown in FIG. 16A according to various embodiments.

FIG. 17A, FIG. 17B, FIG. 17C, and FIG. 17D illustrate various views of an adjustable headset according to various embodiments.

FIG. 18 illustrates a side view of a headset adapted to provide coverage with respect to multiple acoustic windows of a head according to various embodiments.

FIG. 19A, FIG. 19B, FIG. 19C, and FIG. 19D illustrate side views of headsets configured to cover different acoustic windows according to various embodiments.

FIG. 20A, FIG. 20B, FIG. 20C, and FIG. 20D illustrate various views of a device positioned at different acoustic windows according to various embodiments.

### DETAILED DESCRIPTION

The detailed description set forth below in connection with the appended drawings is intended as a description of various configurations and is not intended to represent the only configurations in which the concepts described herein may be practiced. The detailed description includes specific details for providing a thorough understanding of various concepts. However, it will be apparent to those skilled in the art that these concepts may be practiced without these specific details. In some instances, well-known structures and components are shown in block diagram form in order to avoid obscuring such concepts.

In the following description of various arrangements, reference is made to the accompanying drawings which form a part hereof and in which are shown, by way of illustration, specific arrangements in which the arrangements may be practiced. It is to be understood that other arrangements may be utilized, and structural changes may be made without departing from the scope of the various arrangements disclosed in the present disclosure.

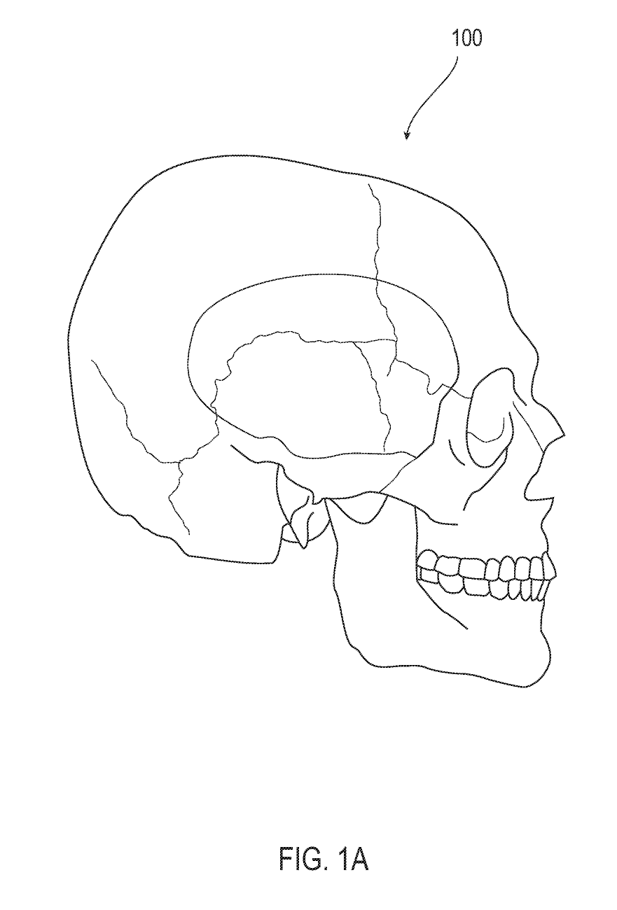

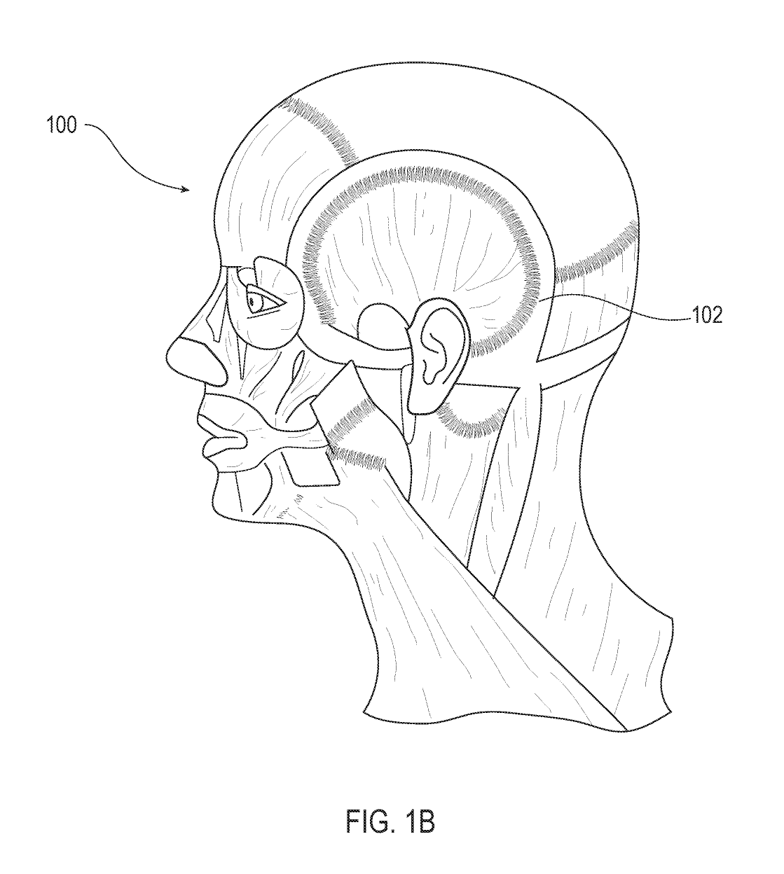

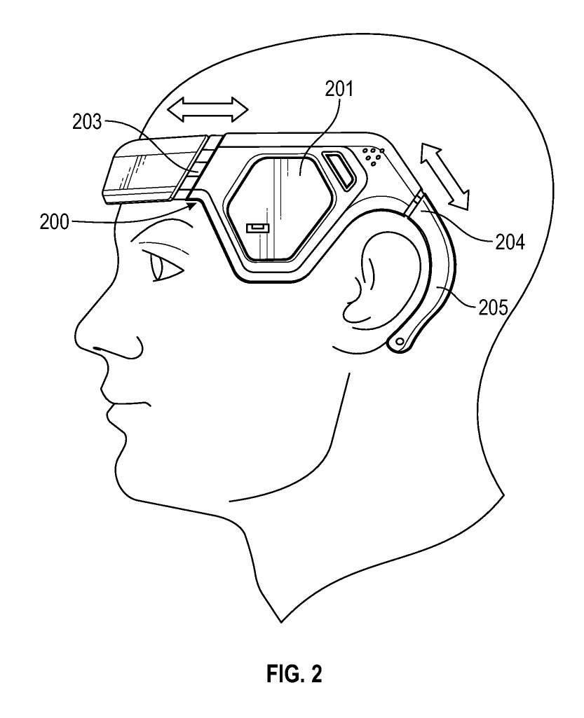

FIG. 1A is a side view of a diagram illustrating the skeletal anatomy of a human head 100. FIG. 1B is a side view diagram illustrating the muscular anatomy of the human head 100.

Referring to FIGS. 1A-1B, the human head 100 includes numerous muscles. However, when a headset including a probe is held fast to a portion of the human head 100 that includes muscle tissue, the headset (and therefore the probe) may shift when a user flexes or uses the muscle tissue. As such, affixing the headset to a muscular portion of the head, or strapping the headset around the entire head, may not be desirable when stabilization and alignment of the probe is a priority. As such, in some arrangements, a headset is designed to be affixed to a portion of a human's head that contains minimal or no muscle so that a probe connected to the headset is not disturbed during operation.

For example, the human head 100 includes a muscular section referred to as the temporalis, which is a fan-shaped muscle that runs from the side of the skull to the back of the lower jaw and is involved in closing the mouth and chewing. Around the temporalis muscular area, the human head 100 includes a bordering ring section referred to as the temporal fascia 102 (shown in FIG. 1B as surrounding the temporalis region). The temporal fascia 102 is an area that has minimal or no muscle, and may include the user's skin and bone only. The temporal fascia 102 includes only bone and skin and begins at the zygomatic process of the frontal bone, continues along the superior and inferior temporal lines on the parietal bone, and further continues around the back of the ear on the mastoid portion (above the mastoid process) of the temporal bone.

According to some arrangements, a headset is held fast to the temporal fascia 102 of a user so that muscular activation or flexing by the user will not disturb the positioning of a probe attached to the headset. Examples of probes suitable for attaching to the disclosed headset are provided in U.S. patent application Ser. No. 15/187,397 and U.S. patent application Ser. No. 15/399,735, which disclosure is incorporated herein by reference. The temporal fascia 102 provides a stable location for holding a headset to a user's head, as the temporal fascia 102 includes minimal or no muscle tissue. In addition, in some arrangements, because the area of the temporal fascia 102 is primarily a boney structure (e.g., between the superior and inferior temporal lines shown in FIG. 1A), a headset secured to this area provides increased comfort to a user, as the headset applies pressure to bone rather than soft tissue and muscle.

In addition, in some arrangements, a headset can be held fast to the galea aponeurotica region of the head, which is a tough fibrous band that extends over the cranium. The galea aponeurotica also provides a stable location for minimizing movement of the headset and probe during operation. In some arrangements, adjustment of the headset focuses pressure on the galea aponeurotica (e.g., adjustment of the headset downwards into the user's skull). In some arrangements, the headset is held fast to the galea aponeurotica in addition to being held fast to the temporal fascia 102. In other arrangements, the headset is held fast to the galea aponeurotica instead of to the temporal fascia 102. In yet other arrangements, the headset is held fast to the temporal fascia 102, but not to the galea aponeurotica. In further arrangements, a rear strap of the headset is included and tracks, without touching, or contacts the external occipital protuberance on the occipital bone.

Accordingly, in various arrangements, a headset is configured to be held fast to a user's head so as to avoid the muscular portions of the head that can shift the headset during operation. For example, in some arrangements, a headset is configured to avoid being held fast to muscles of a user, such as, but not limited to, temporalis (temporal fossa), orbicularis oculi, frontal belly, occipital belly, facial muscles, and neck muscles (e.g., masseter sternocleidomastoid, trapezius, and capitis).

FIG. 2 illustrates a side view of an adjustable headset 200 configured for telescoping adjustment according to various arrangements.

In some arrangements, the headset 200 includes a predominantly C-shaped or U-shaped band (e.g., as viewed from an overhead perspective of the headset 200) that is attached or affixed to a subject's head in horizontal or vertical orientations, such as at the front, back, or top of the head. One drawback of traditional headsets is that they contact (e.g., encircle) and tighten around much of the head so that once attached, the headset contacts and is affected by movement of numerous muscles (e.g., those shown in FIG. 1B). Accordingly, movement of any one muscle contacted by the headset can result in movement of the entire headset, resulting in poor overall stability. Furthermore, if the headset is attached to a device whose performance is optimized by remaining stable with respect to a user's head (e.g., optical devices, surgical devices, scanning devices, medical diagnostic devices, automated Transcranial Doppler devices, and so on), abrupt movement of the device via the attached headset may compromise the accuracy of readings made by the device during its operation.

As such, in some arrangements, the substantially U-shaped or C-shaped headset 200 reduces the number of contact locations between the headset 200 and the human head, and therefore provides less opportunity for the headset 200 to shift or move once affixed to the human head. Accordingly, a device attached to the headset 200 may provide more accurate or precise readings during operation. In some arrangements, the headset 200 includes additional straps for securing the headset 200 to human head.

In various arrangements, the headset 200 is made from any suitable rigid material that maintains its shape. For example, in some arrangements, the headset 200 is made from a material, such as, but not limited to, hard plastic, metals, aluminum, steel, titanium, magnesium, various alloys, rigid plastics, composites, carbon fiber, fiber glass, expanded foam, compression molded foam, stereolithography (SLA) or Fused Deposition Modeling (FDM)-made materials, Reaction Injection Molding (RIM) molding, acrylonitrile butadiene styrene (ABS), thermoplastic olefin (TPO), nylon, polyvinyl chloride (PVC), fiber reinforced resins, or the like.

In some arrangements, further stability is achieved by the headset 200 being designed to squeeze the subject's head, for example, by utilizing padding that is placed at an inner surface of the headset 200 such that the padding is pressed against the head or hair of the subject when the headset 200 is worn. In some arrangements, the headset 200 includes ear hooks or temple tips that are configured to squeeze the bony area (e.g., mastoid process) behind the head.

In some arrangements, the headset 200 includes a device 201 attached thereto (e.g., a probe unit). In some arrangements, the probe unit 201 includes an ultrasound probe that performs ultrasound scans with respect to a subject's head, and the scans include, but are not limited to, Transcranial Doppler (TCD) or Transcranial Color Doppler Sonography (TCCS) with Contrast-Enhanced Transcranial Color Doppler Sonography (CE-TCCS). In some arrangements, the probe unit 201 is configured to be manually operated by an operator. In other arrangements, the probe unit 201 includes a robotic assembly that can move a probe independent of positioning of the headset 200. In some arrangements, the headset 200 is configured to hold the probe unit 201 over and aligned with the general temporal window region of the subject. However, in other arrangements, the headset 200 is configured to hold the probe unit 201 over other regions of the head having different ultrasound windows, as further described below. In various arrangements, the probe unit 201 is a singular probe, an assembly, or an array of probes. In various arrangements, the probe unit 201 is an electrical unit, such as, but not limited to, a phased array antenna system capable of beam steering.

In various arrangements, the C-shaped or U-shaped headset 200 has a predetermined spring constant so that it remains clamped on the subject's head at a desired location. For example, in some arrangements, a default width of the headset 200 is less than a width of a subject's head, but the width of the headset 200 can be elongated by widening it such that the spring constant imparts an inward force on the head once the headset 200 is placed thereon. Accordingly, in various arrangements, the headset 200 is configured as a preset size unique to a subject's head shape and simply slides on to the head such that the spring constant of the headset 200 provides the clamping force. In some arrangements, the spring constant of the headset 200 is adjustable to increase or decrease the distance between the C-shaped or U-shaped band and the head to provide better fit.

In some arrangements, the headset 200 includes a temple adjustment mechanism 203 (e.g., a telescoping temple adjustment mechanism) and a telescoping tip adjustment mechanism 204. In some arrangements, the telescoping temple adjustment mechanism 203 enables the headband section of the headset 200 to extend or contract for a desired fit. Additionally, in some arrangements, the telescoping tip adjustment mechanism 204 enables a temple tip 205 to be adjusted around the ear. Temple tip 205 is configured to encircle the ear to provide additional stability to the headset 200. In some arrangements, one or more of the temple adjustment mechanism 203 and the telescoping tip adjustment mechanism 204 are configured to slide by any suitable mechanism, including but not limited to, a track, a railing, friction fitting, and so on.

In some arrangements, the probe of the device 201 includes an ultrasound probe, and other probes (e.g., near-infrared spectroscopy (NIRS)), a camera, or other accessory can also be attached. In some arrangements, the probe travels around and along the acoustic window of the subject's head to enable improved transmission of acoustic energy to within the skull.

In some arrangements, the device 201 is modular and can be attached and detached from the headset 200. In some arrangements, the headset 200 is used in conjunction with a medical device for use with respect to a user's head (e.g., an ocular monitoring system, a breathing device, a device for monitoring neurological activity, a surgical device, a device for monitoring radioactive traces, or any other device used with respect to a subject's head). In other arrangements, the headset 200 is used in conjunction with a non-medical device for use with respect to a user's head (e.g., a virtual reality eyepiece).

In some arrangements, the device (or probe unit) 201 includes a probe and robotics for controlling the probe. For example, the robotics are configured to translate the probe along a surface of a head and to move the probe towards and away from the head. In some arrangements, an end of the probe interfaces with the robotics, and the robotics include components, such as, but not limited to, a motor assembly and the like for controlling the probe (e.g., control z-axis pressure, normal alignment, or the like of the probe).

In some arrangements, the probe includes a first end and a second end that is opposite to the first end. In some arrangements, the first end includes a concave surface that is configured to be adjacent to or contact a scanning surface (e.g., a head of a subject). The concave surface is configured with a particular pitch to focus generated energy towards the scanning surface. In some arrangements, the device 201 is a TCD apparatus such that the first end of the probe is configured to be adjacent to or contact and align along a human head (e.g., a side of the human head), and the first end of the probe is configured to provide ultrasound wave emissions from the first end and directed into the human head (e.g., towards the brain). In other arrangements, the probe is configured to emit other types of waves during operation, such as, but not limited to, infrared, x-rays, or the like.

In some arrangements, the second end of the probe is coupled to the robotics. In some arrangements, the second end of the probe includes a threaded section along a portion of the body of the probe, and the second end is configured to be secured in the robotics via the threads (e.g., by being screwed into the robotics). In other arrangements, the probe is secured in the robotics by any other suitable connecting means, such as, but not limited to, welding, adhesive, one or more hooks and latches, one or more separate screws, press fittings, or the like.

In other arrangements, the probe is attached within the headset 200 without any robotics, such that the probe is configured to be manually operated by an operator while the headset 200 is positioned on a subject's head. For example, a subject's head can be placed in the headset 200 and an operator can manually shift and orient the probe while the probe is activated.

Further disclosure regarding probe systems that can be used in conjunction with the headsets described herein can be found in non-provisional patent application Ser. No. 15/399,648, titled ROBOTIC SYSTEMS FOR CONTROL OF AN ULTRASONIC PROBE, and filed on Jan. 5, 2017, which is incorporated herein by reference in its entirety. In addition, the description above regarding the device or probe unit 201 is applicable to any of the headsets, devices, or probe units described herein.

In some arrangements, the headset 200 holds other medical and non-medical devices that are used and stabilized with respect to a subject's head. For example, in some arrangements, an ocular device is a device that is optimized by maintaining positioning and alignment with a user's eyes (e.g., if the ocular device is shifted with respect to a user's eyes, performance of the ocular device may decline). In some arrangements, the ocular device is attached at the headset 200 so as to cover the eyes of a patient. As an example of a non-medical device use with respect to the headset 200, in some arrangements, the headset 200 can be used in connection with the ocular device that is a virtual reality device configured to provide a virtual experience to the subject such that any disturbance of the positioning of the ocular device in front of the user's eyes may cause a degradation in the user's virtual experience.

In some arrangements, the ocular device is a medical device designed to track ocular behavior of a subject (e.g., to diagnose whether the user has experienced a concussion). In other arrangements, the ocular device is an ocular diagnosis or treatment tool for determining or adjusting vision of the user. As an example, the ocular device is a device for correcting imperfect vision of a user (e.g., laser eye surgery). As another example, in some arrangements, the ocular device is an ocular diagnostic tool for determining a vision prescription of a user, presence of one or more eye conditions (e.g., glaucoma, cataracts, ocular hypertension, uveitis, or the like), and so on. In some arrangements, the ocular device is designed to cover and interact with both eyes simultaneously or in sequence. In other arrangements, the ocular device is designed to cover and interact with a single eye (e.g., while the other eye remains uncovered). The ocular device can be provided with any of the headset apparatuses described herein.

In some arrangements, more than one device 201 is attached to the headset 200 and aligned with anatomical features. In some arrangements, the device 201 includes the robotics that are configured to move a probe. In some arrangements, the probe includes a camera, NIRS, or an ultrasound emitting device. In some arrangements, the headset 200 provides the ability to adjust the angle and location of the device 201. In some arrangements, an acoustic window is located at the temporal window. In other arrangements, the acoustic window includes the orbital window, the occipital window and/or mandibular window, and the headset 200 is correspondingly designed to allow the device 201 to access these various acoustic windows. For example, instead of the acoustic window being accessible from the temporal window, the headset 200 can provide an opening at the occipital acoustic window for allowing the device 201 access thereto. In various arrangements, the description above with respect to the headset 200 and the device 201 is applicable to any of the headsets described herein.

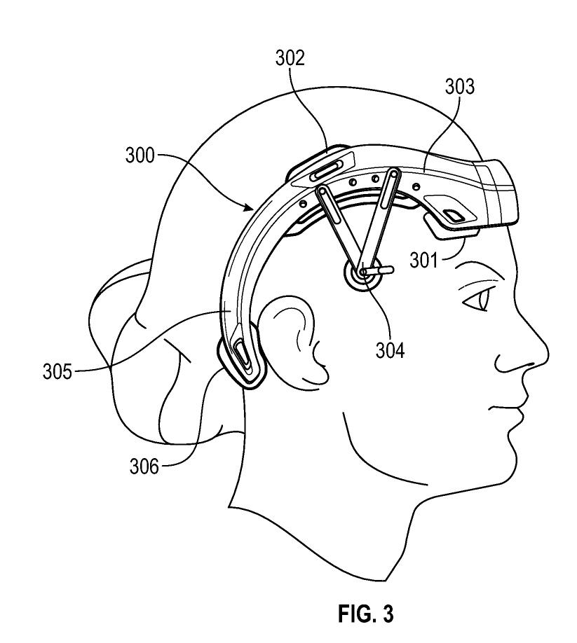

FIG. 3 illustrates a side view of an adjustable headset 300 including a temple tip 301 and a footpad 302 according to various arrangements.

In some arrangements, the headset 300 includes a U-shaped band 303. In some arrangements, the footpad 302 is configured to contact the head of the subject. In particular arrangements, each side of the headset 300 includes a footpad (e.g., the footpad 302). In some arrangements, the footpad 302 is configured to contact areas of the head of the subject having minimal muscle, as described above (e.g., the temporal fascia 102 of the head). In some arrangements, the headset 300 further includes a device 304 (e.g., a probe unit 304) attached thereto. In some arrangements, the probe unit 304 includes an ultrasound probe (e.g., a manually-operated ultrasound probe), a robotically-controlled probe, or a phased array. In particular arrangements, the probe unit 304 includes two adjustable arms connected to the probe of the probe unit 304 to provide freedom of motion of the probe, whether the probe unit 304 is manually operated or automatically controlled. In some arrangements, the headset 300 includes a plurality of mounting points for receiving ends of the adjustable arms along the frame of the headset 300.

In various arrangements, any of the headsets and devices described herein can include a robotic mechanism for automatically controlling a probe or can include a probe configured to be manually controlled by a human.

In some arrangements, the headset 300 includes a strap that is soft and that stretches over the top of the head of the subject. In some arrangements, the headset 300 includes a clear polycarbonate frame that is lifted off the surface of the head by footpads (each of which may be a footpad such as, but not limited to, the footpad 302). In some arrangements, the headset 300 includes a footpad (such as, but not limited to, the footpad 302) at a front of the headset 300 and at a rear of the headset 300.

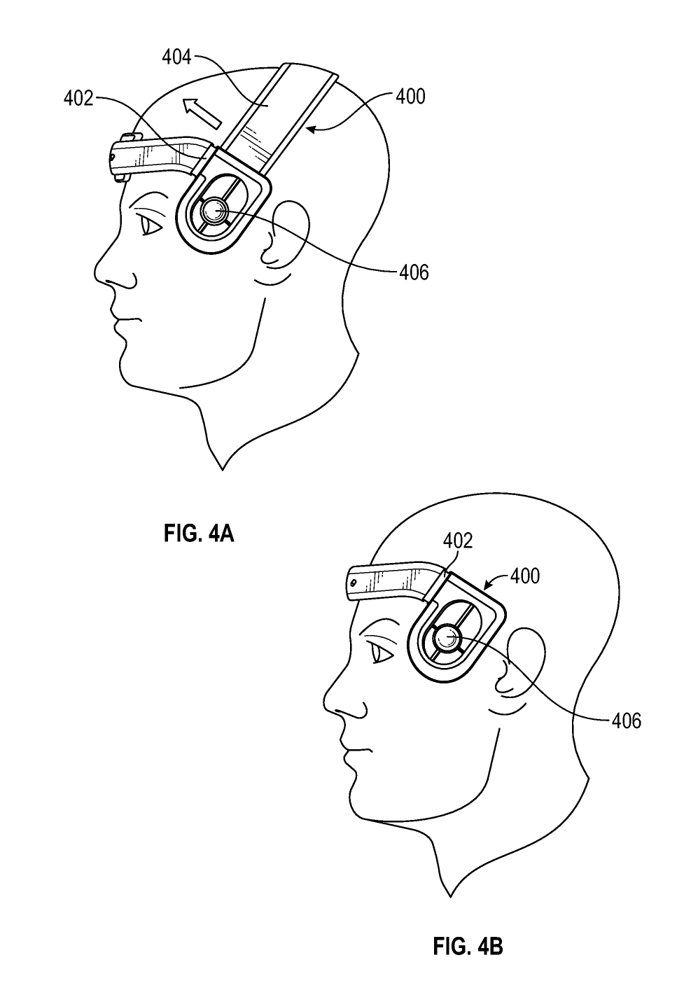

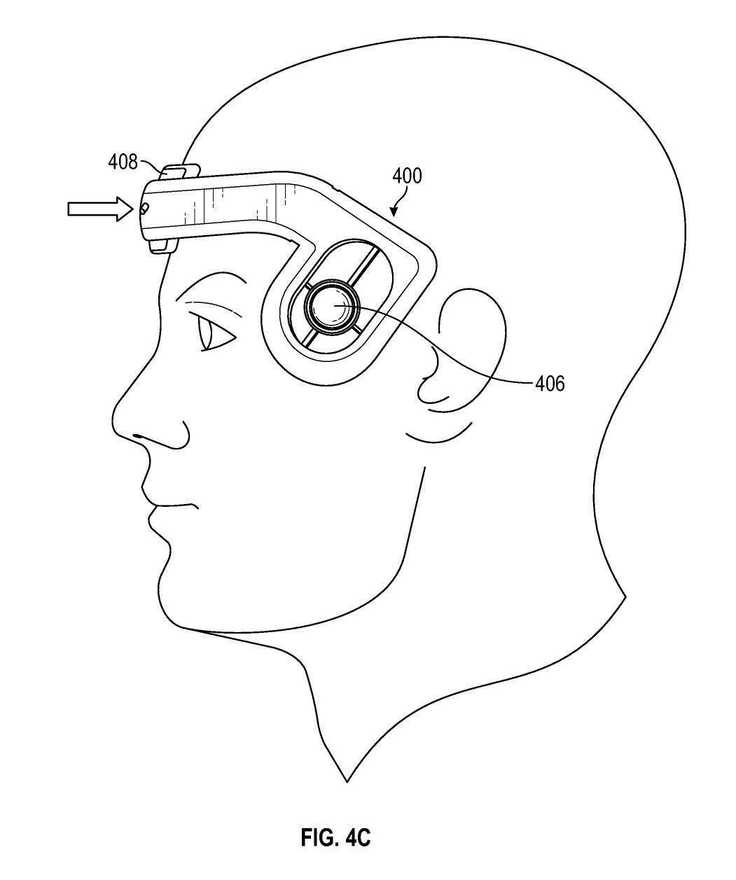

FIG. 4A illustrates a side view of an adjustable headset 400 including a telescoping angled headband 402 and a head strap 404 according to various arrangements. In some arrangements, the telescoping angled headband 402 is at an angle other than parallel to a device (e.g., probe unit) 406, which allows the front band of the headset 400 to be placed at different locations on the forehead of the subject, thereby providing additional degrees of freedom for adjustment of the headset 400. In various arrangements, the head strap 404 (e.g., which is rigid or non-rigid) is used as extra support on the top or back of the head. In some arrangements, the telescoping angled headband 402 allows a location of the device 406 to be adjusted and allows the location of the head strap 404 to be adjusted along a top of the head.

In some arrangements, the head strap 404 is tightened or loosened depending on desired fit of the headset 400 and is held in place with a strap lock. In some arrangements, the strap lock includes, but is not limited to, Velcro, a ratcheting strap, a hook and loop, or any other suitable locking mechanism. In some arrangements, an additional strap (e.g., similar to the strap lock) is held across the back of the head for added stabilization of the headset 400. In various arrangements, the head strap 404 is incorporated into any of the headsets described herein. FIG. 4B illustrates a side view of the adjustable headset 400 shown in FIG. 4A without the head strap 404 according to various arrangements.

FIG. 4C illustrates a side view of the adjustable headset 400 shown in FIG. 4B including an adjustable pad 408 according to various arrangements.

In some arrangements, the adjustable pad 408 is configured to increase or decrease a distance between the frame of the headset 400 (e.g., the front portion of the frame) and the head (e.g., the forehead) of the subject for a desirable fit. In various arrangements, the adjustable pad 408 is made from a soft material, such as, but not limited to, silicone, foam, spring-loaded pad, and the like for improving the comfort of the subject. In some arrangements, the adjustable pad 408 can be removed from the headset 400 (e.g., to be exchanged with another pad) using a quick release mechanism so that different widths of adjustable pads 408 can be used to adjust the distance between the frame of the headset 400 (e.g., the front portion of the frame) and the head (e.g., the forehead) of the subject. Accordingly, in some arrangements, different adjustable pads 408 are designed to have different shapes and sizes (e.g., different thicknesses) to adjust the fit of the headset 400 on the head. In some arrangements, the adjustable pads 408 are disposable such that a new adjustable pad 408 is used after use of the headset 400 for a new subject (e.g., to increase cleanliness and hygiene of the headset 400).

In some arrangements, the adjustable pad 408 is an integrated pad affixed to the headset 400 and for contacting the subject's head. In some arrangements, the adjustable pad 408 is made from any suitable soft material, such as, but not limited to, closed cell foam, open cell foam, self-skinning open or closed cell foams, cast, aerated, or extruded silicone or urethane, polyurethane gels that are configured to distribute pressure efficiently, or the like. In some arrangements, the adjustable pad 408 has any suitable firmness for supporting a head, such as, but not limited to, in a range of about 0.1 pound per square inch (psi) to about 60 psi (e.g., in a range of about 0.1 psi to about 10 psi) or within other suitable ranges of firmness. In some arrangements, the adjustable pad 408 has memory for expanding to fit contours of a head. In some arrangements, the adjustable pad 408 (e.g., foam) is compressed and expands after a user's head is placed against the adjustable pad 408 so that it expands to secure the headset 400. In some arrangements, the headset 400 is manufactured by any suitable process for affixing the adjustable pad 408 within the headset 400, such as, but not limited to, injection molding, laminating, adhesive mounting (e.g., gluing or bonding), co-molding, co-casting, injection, snapping, by Velcro fastening, by hook and loop fastening, friction fitting, attaching with barbs, using screw bosses, or the like.

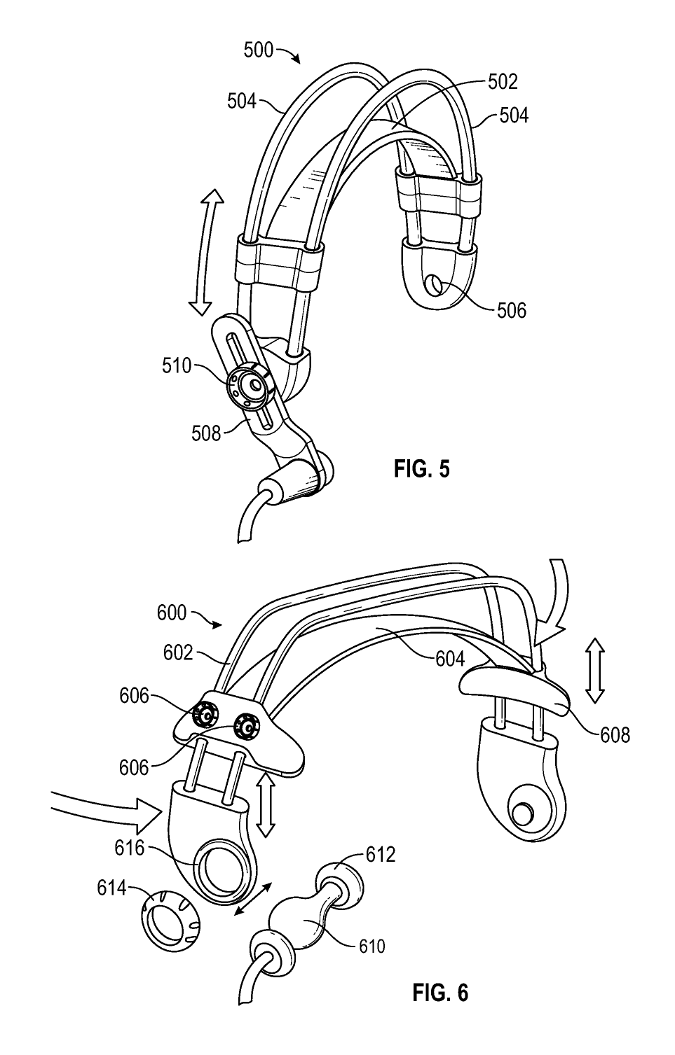

FIG. 5 illustrates a perspective view of an adjustable headset 500 including a spring arm overhead strap 504 according to various arrangements.

In some arrangements, the spring arm overhead strap 504 includes one or more spring arm overhead straps or one or more spring-loaded bands that hold the headset 500 in place. In various arrangements, the spring-loaded band 504 is made from any suitable material that has a predetermined spring constant sufficient to clamp and maintain positioning of the headset 500 on the subject while also affording sufficient comfort, such as, but not limited to, metal or plastic. In some arrangements, additional rigid spring-loaded bands 504 are added to increase spring or clamping pressure and therefore increase stability.

In some arrangements, the headset 500 further includes a padded foot 506, which is an anchor point that is configured to be in direct contact with the head of the subject and is made of a material for providing suitable comfort to the subject, such as, but not limited to, foam or silicone. In some arrangements, the padded foot 506 is also an attachment point so that various devices or accessories can be attached to the headset 500. In some arrangements, a plurality of the padded feet 506 are positioned at discrete locations along the headset 500 such that the padded feet 506 are held fast to different areas of the head of the subject (e.g., areas that have minimal or no muscle), as described above, for providing further stability to the headset 500.

In some arrangements, the headset 500 further includes a sliding head strap 502 that is configured to sit on a top portion of the head of the subject. In some arrangements, the sliding head strap 502 is adjustable vertically along the spring-loaded bands 504 to accommodate different sized and shaped heads. In particular arrangements, the sliding head strap 502 includes two end adjustors at ends of the sliding head strap 502 and that are connected to and surround the spring-loaded bands 504 such that the end adjustors of the sliding head strap 502 uses the bands 504 as a track or railing during vertical adjustment. In further arrangements, the sliding head strap 502 is adjusted along the bands 504 and remains locked in place via friction.

In some arrangements, the headset 500 further includes a device (e.g., probe unit) 508 and a screw adjustment mechanism 510. In some arrangements, the screw adjustment mechanism 510 is configured to be tightened (e.g., to lock a position of the probe unit 508 in place) and loosened (e.g., to adjust positioning of the probe unit 508 or to remove the probe unit 508 from the headset 500 altogether). In other arrangements, the probe unit 508 is connected with the headset 500 via other adjustment mechanisms including, but not limited to, quick locks, snaps, clips, and the like.

FIG. 6 illustrates a perspective view of an adjustable headset 600 including a spring-loaded arm overhead strap 602 and an adjustable head strap 604 according to various arrangements.

In some arrangements, the headset 600 includes one or more screws 606 so that the sliding head strap 604 is locked in place when slid to a desired position along the spring-loaded arm overhead strap 602 (e.g., similar to the headset 500). In some arrangements, a padded foot 608 is directly attached to the adjustable head strap 604. In some arrangements, a ball pivot 610 enables the location of a probe 612 to be adjusted about a travel path. For example, the probe 612 can be manually moved along three or more degrees of freedom. In some arrangements, the ball pivot 610 is held in place by a screw-on cap 614 and a threaded anchor point 616 located on the headset 600.

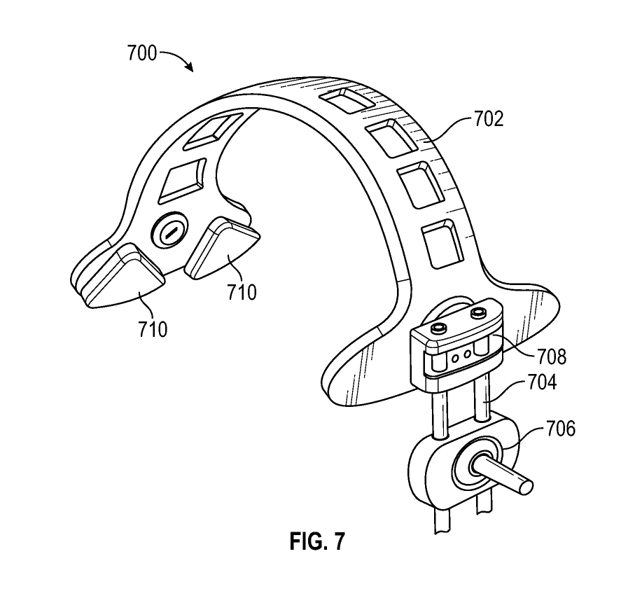

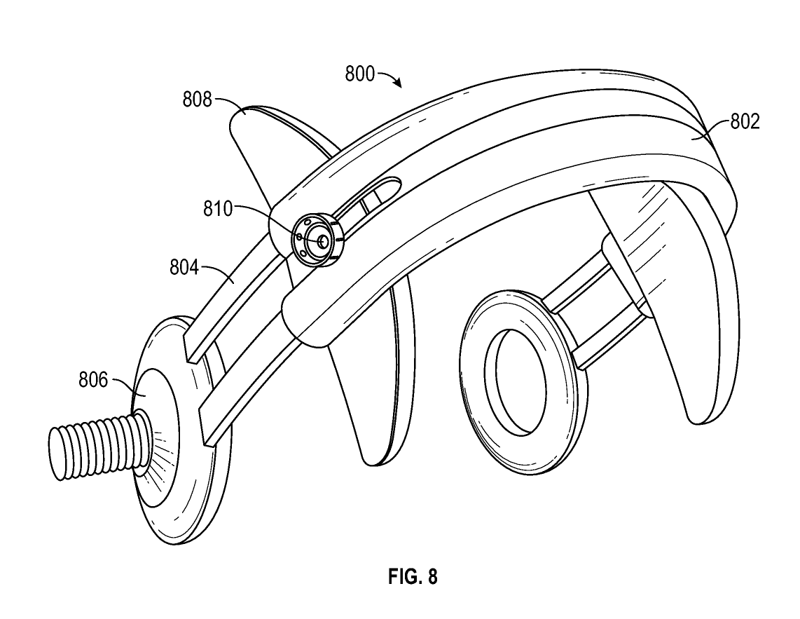

FIG. 7 illustrates a perspective view of an adjustable headset 700 including a spring arm overhead strap 702 and a telescoping probe adjustment support 704 according to various arrangements.

In some arrangements, the headset 700 includes the spring arm overhead strap (e.g., a spring arm overhead U-shaped band) 702 and the telescoping probe adjustment support 704 connected to a device (e.g., probe unit) 706. In some arrangements, the telescoping probe adjustment support 704 is secured with a support locking clip 708. However, in other arrangements, other suitable locking mechanisms, such as, but not limited to, locking screws are implemented. In some arrangements, the headset 700 further includes one or more padded feet 710, as described above.

FIG. 8 illustrates a top perspective view of an adjustable headset 800 including a spring arm overhead strap 802 and a screw-tightened telescoping probe adjustment support 804 according to various arrangements. In some arrangements, the headset 800 includes the spring arm overhead strap (e.g., a U-shaped band) 802 and the telescoping probe adjustment support 804 for a probe unit 806. In some arrangements, the headset 800 further includes a padded foot 808, as described above. In some arrangements, the headset 800 includes one or more screws 810 so that the telescoping probe adjustment support 804 is locked in place when slid to a desired position along the spring arm overhead strap 802.

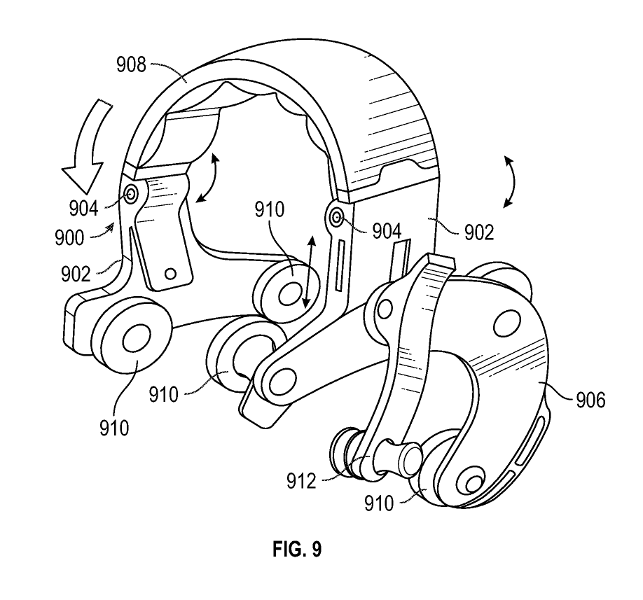

FIG. 9 illustrates a perspective view of an adjustable headset 900 including a spring-loaded foldable arm 902 according to various arrangements. In some arrangements, the headset 900 includes a hinge 904 that enables the spring-loaded foldable arm 902 to fold up and reduce space when not in use, which is desirable when the headset 900 is stored or transported. In some arrangements, the hinge 904 is kept in place during use using an internal tightening mechanism, such as, but not limited to, an internal spring, clamp, or tightening mechanism. In some arrangements, an ear tip 906 is incorporated into the headset 900 to provide further stabilization thereto (e.g., by anchoring the headset 900 around the ear of the subject). In some arrangements, the headset 900 further includes a head band 908, a plurality of feet 910, and a device (e.g., probe unit) 912.

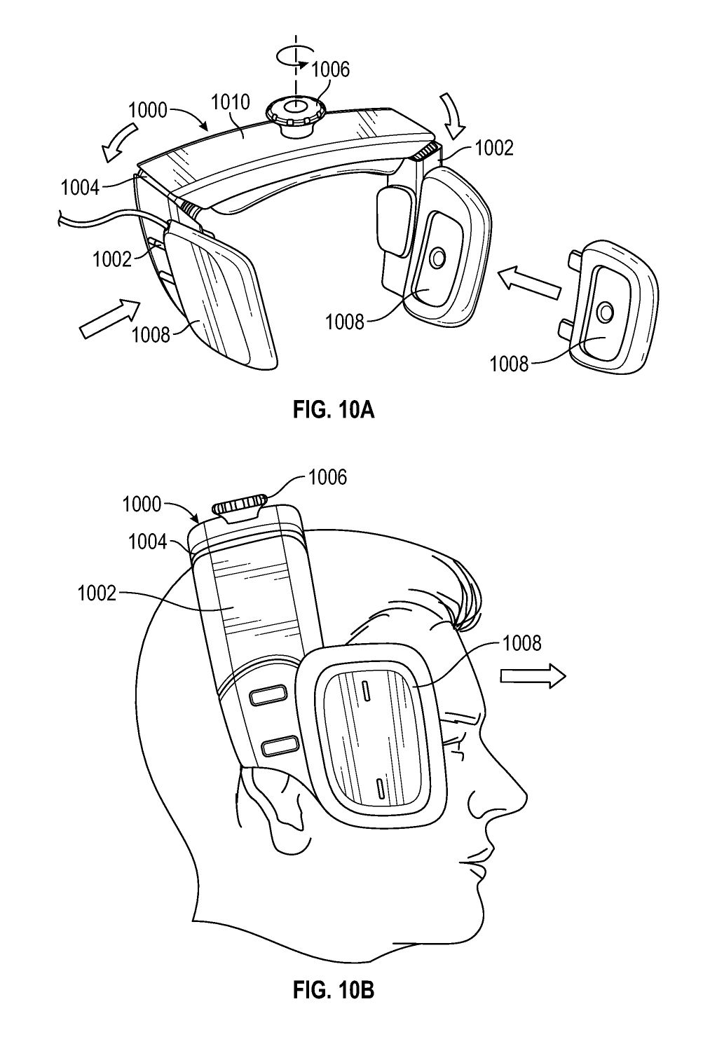

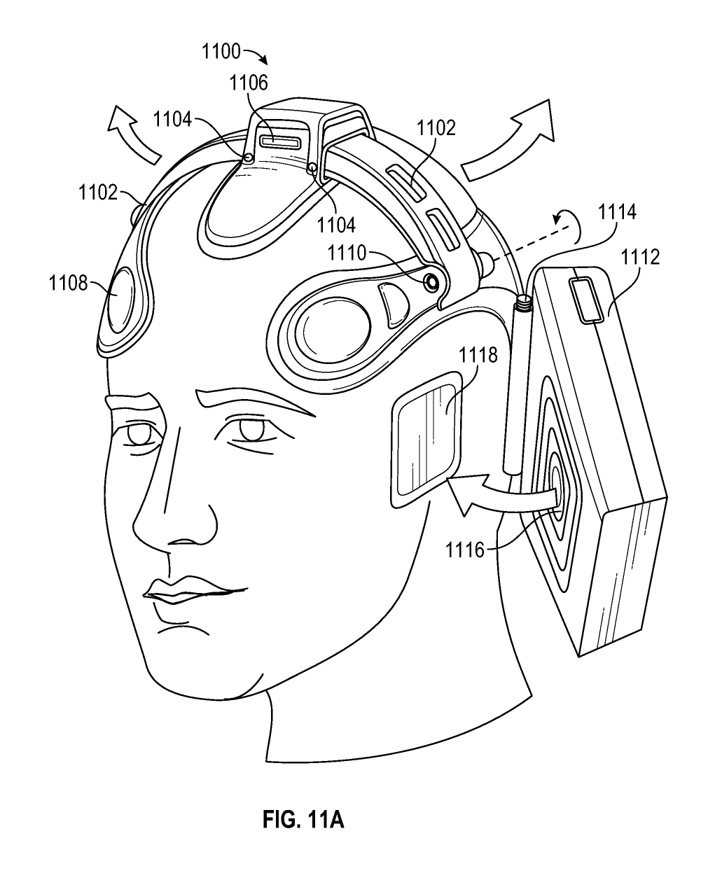

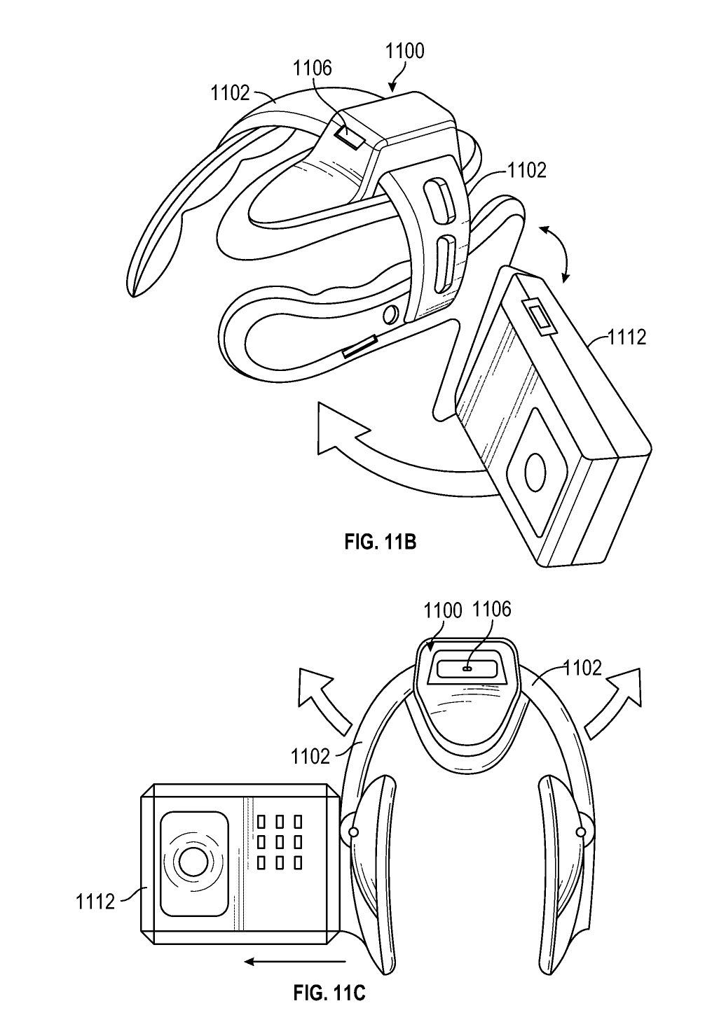

FIG. 10A illustrates a perspective view of an adjustable headset 1000 including a foldable arm 1002 according to various arrangements. FIG. 10B illustrates a side view of the adjustable headset 1000 shown in FIG. 10A according to various arrangements.

In some arrangements, the headset 1000 includes a hinge 1004 that enables the arm 1002 to fold up and reduce space taken up by the headset 1000 when not in use, which is desirable when the headset 1000 is stored or transported. In some arrangements, the headset 1000 includes a folding lock mechanism 1006. In some arrangements, the folding lock mechanism 1006 is used to apply tension to rods connected to the arm 1002 such that the arm 1002 is locked in the unfolded position during use and in the folded position during storage. In some arrangements, the foldable arm 1002 is configured to pivot around the hinge 1004 and tightened into place via the folding lock mechanism 1006. In some arrangements, the folding lock mechanism 1006 is twisted, which tightens an inner screw that, in turn, applies tension to the inner rods connected to the arm 1002 so that when tightened, the rods and thus the arm 1002 is restricted from movement. In other arrangements, other suitable mechanisms of locking the arm 1002 are utilized including, but not limited to, an inner gear mechanism, a wire wrapped around a spindle, and the like. In some arrangements, a device 1008 (e.g., probe unit 1008) is snapped in place on the arm 1002 of the headset 1000 (e.g., as illustrated in FIG. 10A). In some arrangements, the probe unit 1008 is released from the headset 1000 via a quick release button. In some arrangements, the headset 1000 further includes a head band 1010.

FIG. 11A illustrates a perspective view of an adjustable headset 1100 including an adjustable side arm 1102 according to various arrangements. FIG. 11B illustrates a top perspective view of the adjustable headset 1100 shown in FIG. 11A according to various arrangements. FIG. 11C illustrates a front view of the adjustable headset 1100 shown in FIG. 11A according to various arrangements.

In some arrangements, the headset 1100 includes the adjustable side arm 1102 that adjusts outwards and inwards using a swinging motion. In some arrangements, the side arm 1102 swings about a pivot point 1104, which enables a user to adjust the headset 1100 from a folded-up state to a deployed state (e.g., into a U-shape) that is configured to fit different head shapes. In some arrangements, a locking mechanism 1106 is used to tighten or loosen the side arm 1102. In some arrangements, the locking mechanism 1106 (e.g., when pressed in) disengages a wedge-shaped snap that is otherwise held in an engaged position with a spring and thereby enables movement of the side arm 1102. In other arrangements, different mechanisms are used to lock and enable motion of the side arm 1102, such as, but not limited to, by actuating a receiver to disengage from a pin located on the side arm 1102.

In some arrangements, the headset 1100 includes more than one side arm 1102. For example, a side arm 1102 can be located at each side of a subject's head, at a back of the head (e.g., situated perpendicularly to the U-shaped headset 1100), at a front of the head, and so on (e.g., to add stability and support by contacting additional areas of the head of the subject). In some arrangements, one locking mechanism 1106 is used at each side arm 1102 to thereby control locking of the side arms 1102 independently from each other.

In some arrangements, a foot structure 1108 is contoured to fit a human head. For example, the foot structure 1108 wraps along a significant portion of either the superior temporal line or the inferior temporal line and has a suitable shape to maintain contact along the circumference of the head. In some arrangements, the foot structure 1108 is a rotating foot structure that rotates about a pin 1110 (or a pivoting foot structure that rotates about a ball joint). In some arrangements, the foot structure 1108 is locked into position or restricted from pivoting motion by tightening a knob connected to the pin 1110. In some arrangements, the foot structure 1108 includes a spring mounted foot mechanism that has a spring constant that maintains suitable pressure against the head and a foam padded mount having inner contours. In some arrangements, pads of the foot structure 1108 include a soft material, such as, but not limited to, foam, gel pads, removable padding, and the like.

In some arrangements, a device (e.g., probe unit) 1112 is attached to the headset 1100 via a probe unit hinge 1114, which enables swinging motion of the probe unit 1112. In some arrangements, an operator locks the probe unit 1112 in place with respect to the subject. In some arrangements, the probe unit 1112 includes a probe 1116 (e.g., an ultrasound probe). In some arrangements, the probe unit 1112 includes a fully automated robotic unit capable of translating the probe along one or more axes. For example, the probe can be moved in the X, Y, and Z axes, as well as movement by panning or tilting. In some arrangements, the probe unit 1112 is positioned so that the probe 1116 can be translated about a temporal window 1118. In some arrangements, the probe unit 1112 includes an ultrasound probe that performs ultrasound scans with respect to a subject's head, and the scans include, but are not limited to, Transcranial Doppler (TCD) or Transcranial Color Doppler Sonography (TCCS) with Contrast-Enhanced Transcranial Color Doppler Sonography (CE-TCCS). In other arrangements, other probes, such as, but not limited to, phased arrays are used.

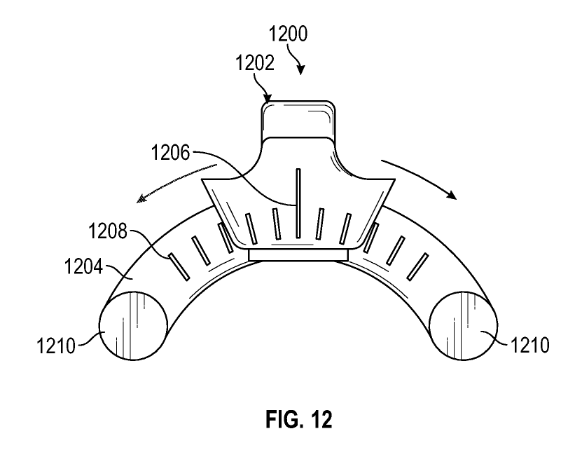

FIG. 12 illustrates a side view of an adjustable headset 1200 configured to provide adjustment along a radius according to various arrangements.

In some arrangements, the adjustable headset 1200 is configured to be adjusted along a radius of the headset 1200 such that a U-shaped strap 1202 can be placed at a suitable position along the top of the head (e.g., anywhere from the forehead region of the head to the occipital bone region of the back of the head), while maintaining the same position of a foot pad support 1204 near the temporal window of the user. In some arrangements, the position of the U-shaped strap 1202 can be adjusted using a screw adjustment 1206 that moves a gear within the headset 1200 along a plurality of notches 1208 in the foot pad support 1204. However, in other arrangements, different locking mechanisms are used to maintain positioning of the U-shaped strap 1202, such as, but not limited to, a locking screw that clamps down on the foot pad support 1204 and the like. In some arrangements, the screw adjustment 1206, the notches 1208, and the foot pad support 1204 are made from any suitable rigid material, such as, but not limited to, metal, hard plastic, metals, aluminum, steel, titanium, magnesium, various alloys, rigid plastics, composites, carbon fiber, fiber glass, expanded foam, compression molded foam, SLA or FDM-made materials, RIM molding, ABS, TPO, nylon, PVC, fiber reinforced resins, or the like.

In some arrangements, a z-axis distance (e.g., a depth distance along an axis going into the head of the subject) between a forward-most mounting foot 1210 (e.g., the mounting foot 1210 that is closest to the forehead of the subject when the headset 1200 is worn) and the rear-most mounting foot 1210 (e.g., the mounting foot 1210 that is closest to the user's ear when the headset 1200 is worn) is between about 70 millimeters (mm) to about 170 mm. In some arrangements, the mounting feet 1210 are aligned so that they contact the region of the subject's head that include the temporal fascia 102 (e.g., as shown in FIG. 1B).

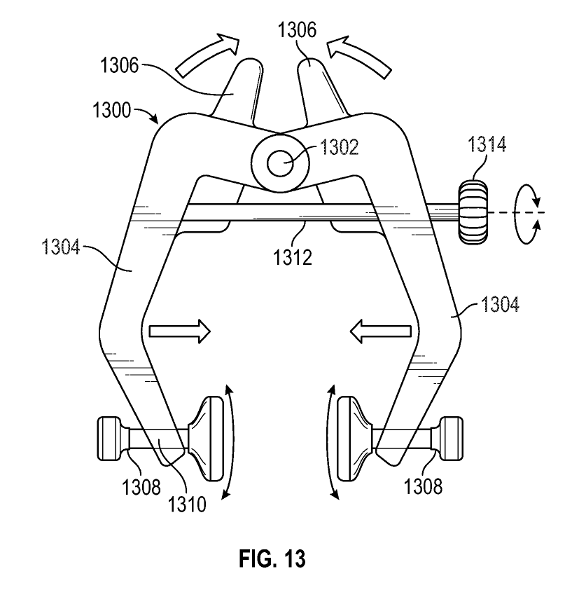

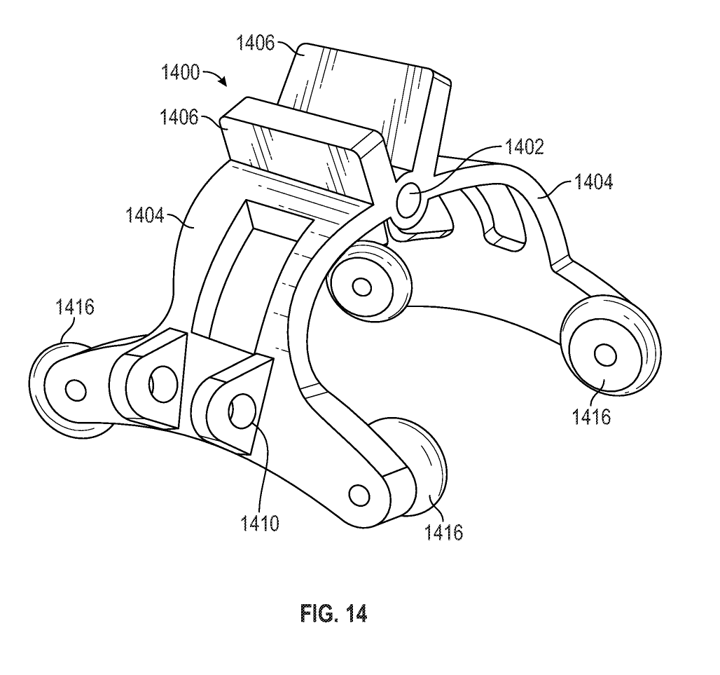

FIG. 13 illustrates a front view of an adjustable headset 1300 including a spring hinge clamp 1302 according to various arrangements.

In some arrangements, the headset 1300 includes two side arms 1304 that are held on a head of a subject via a spring hinge clamp 1302. In some arrangements, the spring hinge clamp 1302 is operated by squeezing a grip 1306 together to open or widen the headset 1300. When the grip 1306 is released, the headset 1300 will close until sufficient resistance is applied thereto (e.g., either by stopping on the head of the subject or folding for storage). In some arrangements, an internal spring is located within the headset 1300, which causes the two side arms 1304 to pull together and tighten around a subject's head.

In some arrangements, a device (e.g., probe unit) 1308 is attached by being screwed directly into the headset 1300 through an anchor point 1310. In other arrangements, the probe unit 1308 is attached via a quick release, a hinge, or any other method. In some arrangements, the probe unit 1308 is an ultrasound probe that is used to perform ultrasound scans within the head, such as, but not limited to, transcranial Doppler (TCD), a transcranial color Doppler sonography (TCCS) with contrast-enhanced transcranial color Doppler sonography (CE-TCCS), or a robotic assembly that can move the probe independent of headset position. In some arrangements, the headset 1300 holds the probe unit 1308 over the general temporal window region. However, in other arrangements, the headset 1300 holds the probe unit 1308 over other regions of the head having ultrasound windows. In various arrangements, the probe unit 1308 is a singular probe, an assembly, or an array of probes. In various arrangements, the probe unit 1308 is an electrical unit, such as, but not limited to, a phased array antenna system capable of beam steering.

In some arrangements, an adjustment mechanism 1312 (e.g., a screw) can be tightened or loosened using a knob 1314, which causes the headset 1300 to close or open. In some arrangements, one or both side arms 1304 are threaded for adjustment mechanisms 1312 (e.g., screws) so that during operation, travel along the screw shaft will cause the side arms 1304 to open or close.

FIG. 14 illustrates a perspective view of an adjustable headset 1400 including a spring hinge clamp 1402 according to various arrangements.

In some arrangements, the headset 1400 is held on a subject's head using the spring hinge clamp 1402 according to various arrangements. In some arrangements, the headset 1400 is designed to include anchor points 1410 so that a device (e.g., probe unit) can be attached therein. In some arrangements, one or more foot pads 1416 contact the subject's head and are designed to increase comfort of the subject and are made from any suitable soft or flexible material described herein. In some arrangements, the foot pad 1416 is designed to provide a sufficient anchor to the subject's head so that unwanted movement of the headset 1400 is minimized during use.

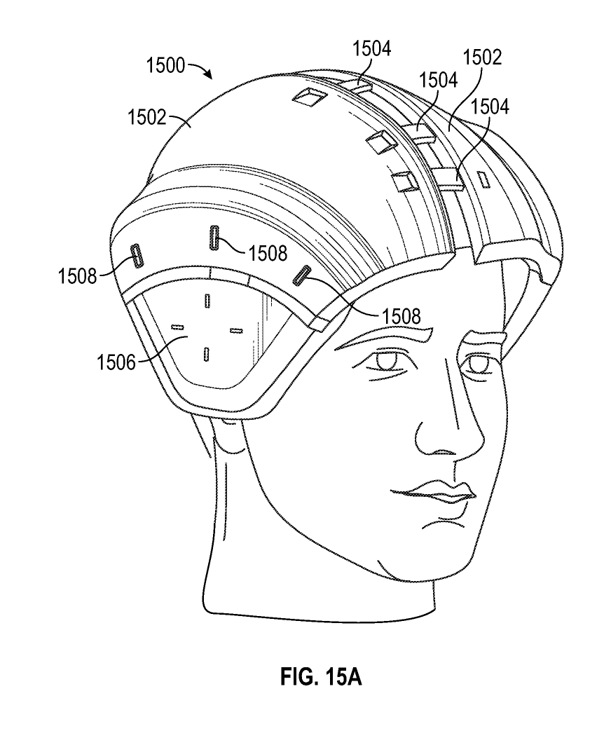

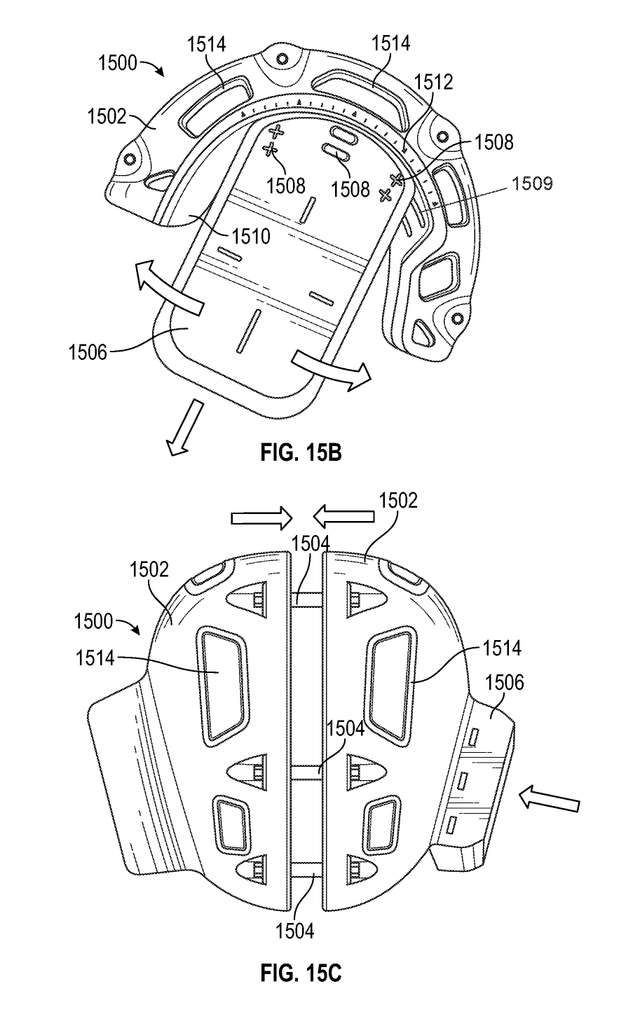

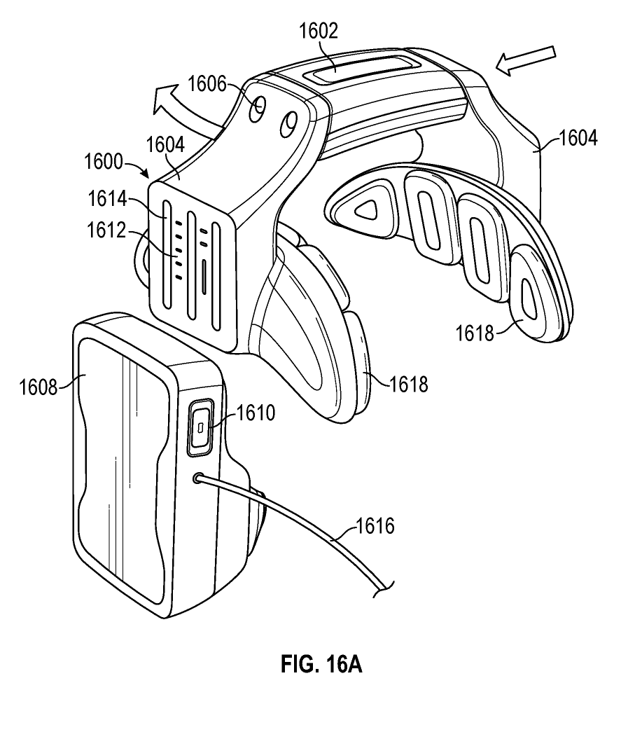

FIG. 15A illustrates a perspective view of an adjustable headset 1500 having two separated sections 1502 according to various arrangements. FIG. 15B illustrates a side view of the adjustable headset 1500 shown in FIG. 15A according to various arrangements. FIG. 15C illustrates a top view of the adjustable headset 1500 shown in FIG. 15A according to various arrangements.

In some arrangements, the headset 1500 is made from any suitable rigid material, such as, but not limited to, metal, hard plastic, aluminum, steel, titanium, magnesium, various alloys, rigid plastics, composites, carbon fiber, fiber glass, expanded foam, compression molded foam, SLA or FDM-made materials, RIM molding, ABS, TPO, nylon, PVC, fiber reinforced resins, or the like.

In some arrangements, the headset 1500 is predominantly shaped as two half sections 1502 that can be attached to a subject's head by being shifted about one or more central adjustment mechanisms 1504. In some arrangements, the half sections 1502 range in size. For example, the headset 1500 can cover an entire side of a head (e.g., similar to half a helmet), or the headset 1500 can have a small, half crescent shape that affixes and is positioned at the temporal fascia 102.

In some arrangements, the central adjustment mechanism 1504 acts as a slide adjustment where the two half sections 1502 can be slid closer or further from each other to fit the shape and size of the subject's head. However, in other arrangements, the adjustment mechanism 1504 is a hinged-based system so that the headset 1500 is folded over the subject's head, the adjustment mechanism 1504 includes rigid gears and strips, the adjustment mechanism 1504 includes rods and bearing slides or ratchet strips with clamps, and the like. In some arrangements, the adjustment mechanism 1504 locks into place so that once the headset 1500 is suitably placed on the subject's head the adjustment and fit of the headset 1500 does not change.

In some arrangements, the headset 1500 includes a probe unit 1506 attached thereto. In some arrangements, the probe unit 1506 includes an ultrasound probe used to perform ultrasound scans within the head of the subject, such as, but not limited to, transcranial Doppler (TCD), a transcranial color Doppler sonography (TCCS) with contrast-enhanced transcranial color Doppler sonography (CE-TCCS), or a robotic assembly that can move the probe independent of headset position. In some arrangements, the headset 1500 holds the probe unit 1506 over the general temporal window region of the subject's head. In other arrangements, the headset 1500 holds the probe unit 1506 over other regions of the subject's head including other ultrasound windows. In various arrangements, the probe unit 1506 is a singular probe, an assembly, or an array of probes. In various arrangements, the probe unit 1506 is an electrical unit, such as, but not limited to, a phased array antenna system capable of beam steering.

In some arrangements, the headset 1500 includes one or more quick-release mechanisms 1508 to facilitate removal of the probe unit 1506. In some arrangements, the quick-release mechanisms 1508 are released by pressing therein such that a locking mechanism is released. In some arrangements, suitable locking mechanisms include, but are not limited to, quick release ball-type locking pins, notches, or a plate and mounting base.

In some arrangements, the headset 1500 includes the two half sections 1502 that are adjusted to fit on a head of a subject by shifting about the central adjustment mechanism 1504. In some arrangements, the probe unit 1506 is further adjustable and can be rotated or moved along a track 1510. In some arrangements, the track 1510 is circular, but can also be straight or any other shape depending on specifications in various arrangements. In some arrangements, an adjustment indicator 1512 includes alignment marks so that the location of the probe unit 1506 can be noted and returned to for future adjustments. In other arrangements, the adjustment indicator 1512 is used on any headset described herein.

In some arrangements, the headset 1500 includes padding 1514 so that the headset 1500 is more comfortable to the subject. In some arrangements, foam or other materials previously described can be used for the padding 1514. In some arrangements, the padding 1514 is shaped to contact specific regions of the head that have minimal or no underlying muscle (e.g., as described with respect to FIGS. 1A and 1B) to reduce movement of the headset 1500 during use.

In some arrangements, the probe unit 1506 is attached using the quick-release mechanisms 1508 (e.g., similar to camera equipment) where a plate is incorporated into the probe unit 1506. In some arrangements, the plate includes a dovetail and varies in length. In some arrangements, the headset 1500 include the track 1509 that acts as a mounting base so that the plate probe unit 1506 can be attached thereto. In some arrangements, the plate is fully opened, allowing it to drop in, or partially opened so that it can slide in. Accordingly, in some arrangements, the probe unit 1506 can slide without need for complete removal thereof. In some arrangements, the plate and base are reversed such that the plate is incorporated into the headset 1500 and the base on the probe unit 1506.

As an example of a quick release system used with respect to the probe unit 1506, the quick release system can be based on a two-piece mechanism. The first piece, generally referred to as a “plate,” is attached to the headset 1500 or to the probe unit 1506. The second piece is the mounting base, generally referred to as a “clamp”, where the plate gets attached and secured. The clamp can be fully opened, allowing the plate to be dropped in and secured, or can be partially opened to allow the plate to be slid into the position. Accordingly, the quick release system according to various arrangement, allows the ability to slide the plate without having to worry about mounting or dismounting anything. In addition, in some arrangements, the plate can have a 45° dovetail thereon, which allows equipment to be moved across the clamp and secured in a specific location using a side knob or a locking release.

In some arrangements, the headset 1500 includes the two half sections 1502 that can be adjusted to fit on the head of the subject by moving or sliding about the central adjustment mechanism 1504. In some arrangements, the central adjustment mechanism 1504 (e.g., linear slide) enables easier positioning of the headset 1500. In some arrangements, any suitable number of linear slides can be used for allowing adjustment of the two half sections 1502. In some arrangements, the headset 1500 includes more than two sections such that there is more than one row of central adjustment mechanisms 1504.

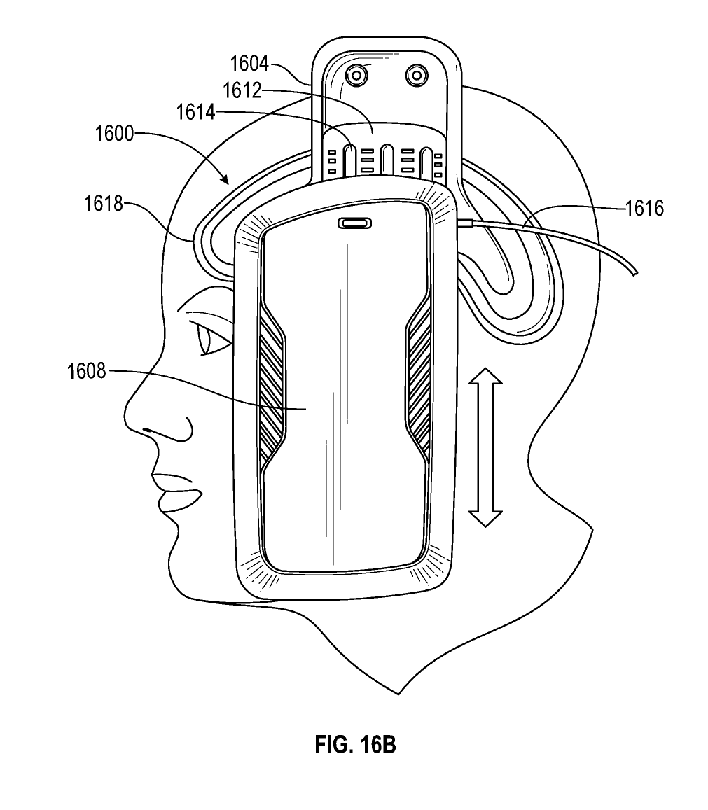

FIG. 16A illustrates a perspective view of an adjustable headset 1600 including a handle grip 1602 according to various arrangements. FIG. 16B illustrates a side view of the adjustable headset 1600 shown in FIG. 16A according to various arrangements.

In some arrangements, the headset 1600 is made from any suitable rigid material, such as, but not limited to, metal, hard plastic, metals, aluminum, steel, titanium, magnesium, various alloys, rigid plastics, rubber, composites, carbon fiber, fiber glass, expanded foam, compression molded foam, SLA or FDM-made materials, RIM molding, ABS, TPO, nylon, PVC, fiber reinforced resins, or the like.

In some arrangements, the headset 1600 is predominantly U-shaped when viewed from the front. In some arrangements, the handle grip 1602 allows an operator to easily carry the headset 1600. In some arrangements, the headset 1600 includes an arm 1604 that is made from a flexible material configured to bend or flex to facilitate suitable fit on a head of a subject.

In some arrangements, the headset 1600 includes one or more adjustment screws 1606 that are used to either adjust flexibility characteristics of the headset 1600 (e.g., through a truss rod within the arm 1604) or to change the distance between each arm 1604. In other arrangements, the adjustment screws 1606 are any other suitable type of tightening mechanism (e.g., instead of screws tightened with external instruments), such as, but not limited to, knobs for tightening the headset 1600 by hand (e.g., knobs that protrude outwards).

In some arrangements, the arm 1604 further provides a spring force against the sides of the head when tightened with the assistance of internal foam pads or spring-loaded feet that offer resistance and spring force on the inside of the headset 1600 against the head. In other arrangements, other mechanisms for providing a clamping force against the subject's head include, but are not limited to, using plastic materials that flex slightly and apply spring force as the adjustment screws 1606 are tightened against the head or by using a left to right adjustment mechanism that is spring loaded that allows the headset 1600 to squeeze the subject's head at a predetermined pressure.

In some arrangements, the headset 1600 includes a device (e.g., probe unit) 1608 that is attachable thereto. In some arrangements, the probe unit 1608 includes an ultrasound probe that can be used to perform ultrasound scans within the head such as transcranial Doppler (TCD), a transcranial color Doppler sonography (TCCS) with contrast-enhanced transcranial color Doppler sonography (CE-TCCS), or a robotic assembly that can move the probe independent of headset position. In some arrangements, the headset 1600 holds the probe unit 1608 over the general temporal window region of the subject's head, but in other arrangements, the headset 1600 holds the probe unit 1608 over other regions of the head that correspond to other ultrasound windows. In various arrangements, the probe unit 1608 is a singular probe, an assembly, or an array of probes. In various arrangements, the probe unit 1608 is an electrical unit, such as, but not limited to, a phased array antenna system capable of beam steering.

In some arrangements, the headset 1600 includes a quick release mechanism 1610 to facilitate simple and easy removal of the probe unit 1608. In some arrangements, the quick release mechanism 1610 is actuated by pressing therein such that a locking mechanism is released. In some arrangements, suitable locking mechanisms include, but are not limited to, quick release ball-type locking pins, notches, or plate and mounting base.

In some arrangements, the probe unit 1608 is attached using a quick release system similar to camera equipment where an attachment mechanism 1612 on the headset 1600 is a base incorporated into the headset 1600 and includes receiving tracks 1614. In some arrangements, the attachable probe unit 1608 includes a plate having a dovetailed extension or similar protruding material which can vary in length. In some arrangements, the receiving tracks 1614 acts as a mounting receiver so that the dovetail of the probe unit 1608 fits therein and facilitates proper alignment for simplifying attachment. In some arrangements, the plate is fully open, allowing it to drop in, or partially opened so that it can slide in. In some arrangements, the probe unit 1608 is slid about the receiving tracks 1614 without complete removal thereof.

In some arrangements, the probe unit 1608 includes a hose or tube 1616 for injection of a lubricating liquid such as ultrasound gel. In various arrangements, any of the headsets including probe units described herein can include such tube or hose. In some arrangements, the end of the probe unit 1608 facing the subject defines an opening that allows the lubricating liquid to excrete therefrom and contact the head of the subject.

In some arrangements, the headset 1600 includes a foam padding 1618 that is shaped to conform to a region of the subject's head originating at the temples and ending at the crown. However, in other arrangements, the foam padding 1618 wraps completely around the head. In some arrangements, the foam padding 1618 is made from the same or similar materials as those of other paddings described herein.

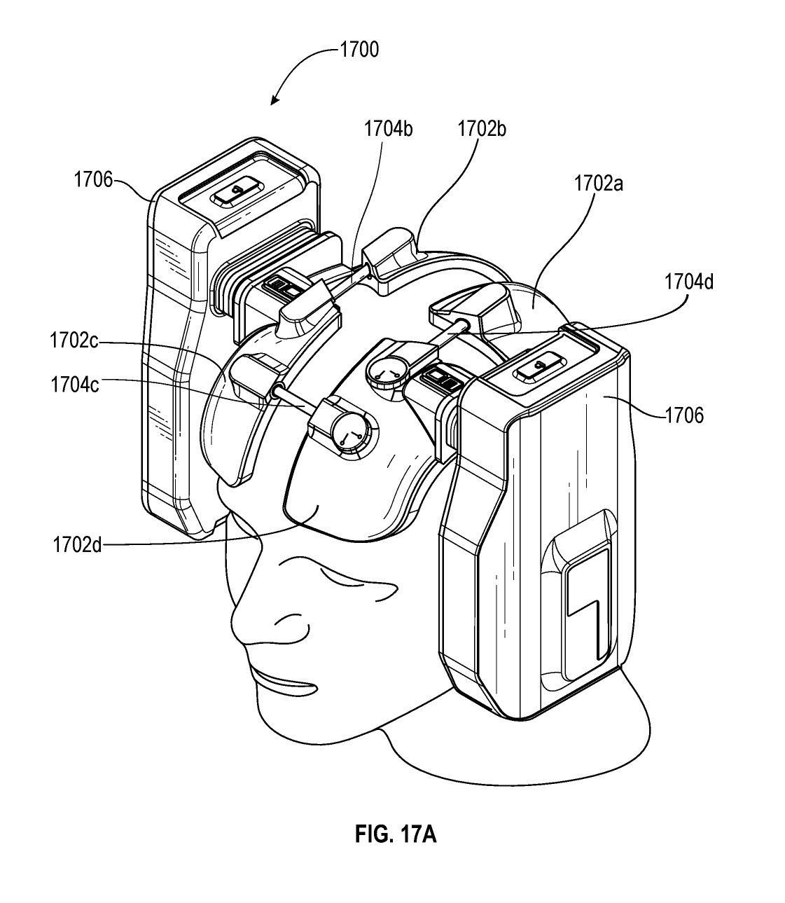

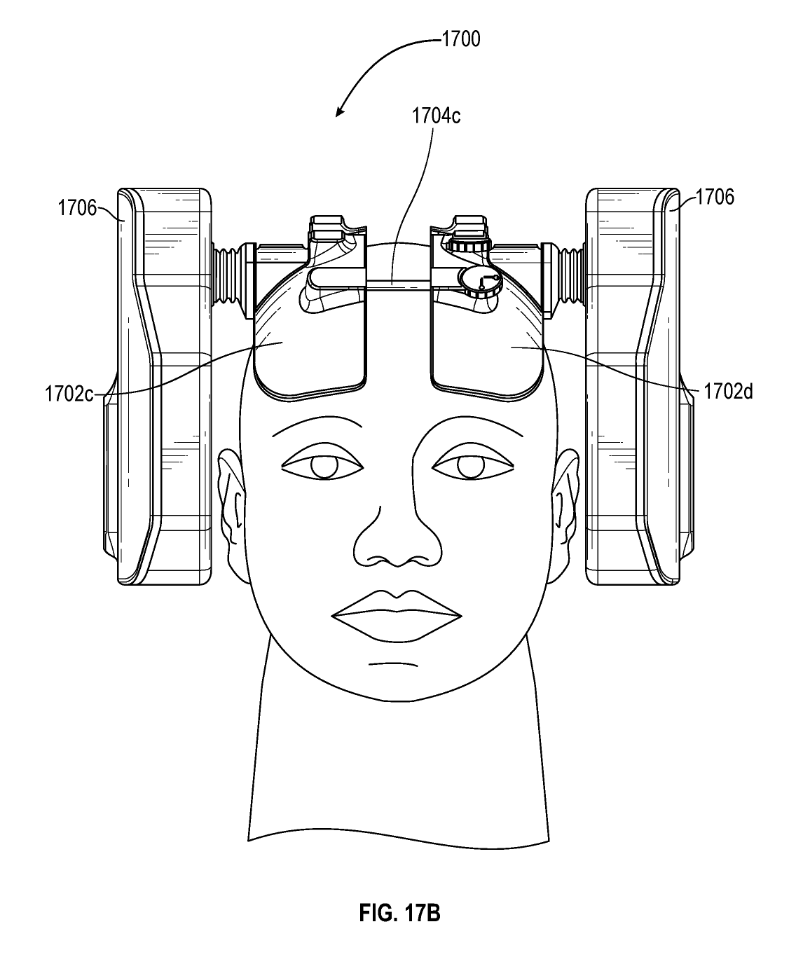

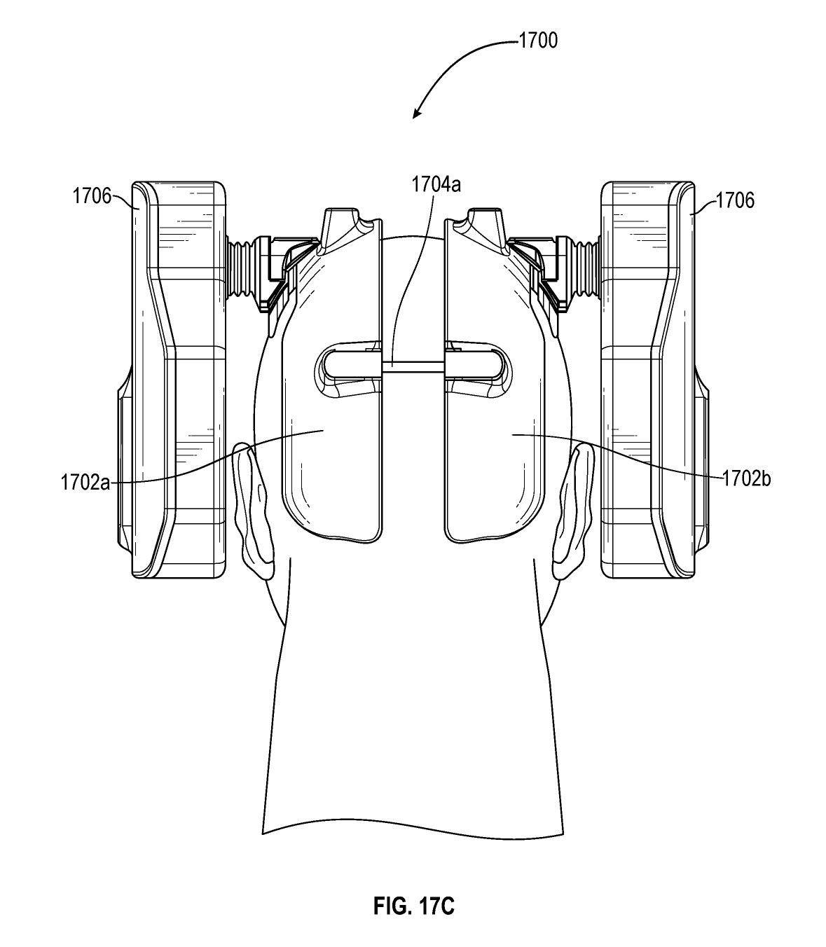

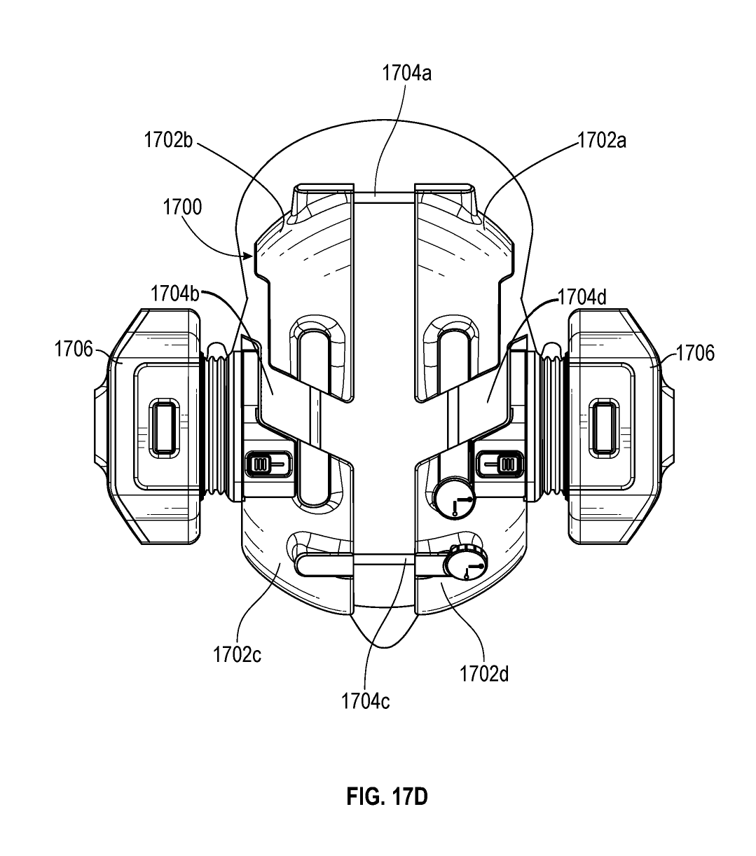

FIG. 17A, FIG. 17B, FIG. 17C, and FIG. 17D illustrate various views of an adjustable headset 1700 according to various arrangements.

In some arrangements, the headset 1700 includes a plurality of head pieces 1702a, 1702b, 1702c, 1702d, a plurality of connectors 1704a, 1704b, 1704c, 1704d between the head pieces 1702a, 1702b, 1702c, 1702d, and one or more devices (e.g., probe units 1706). In some arrangements, the head pieces 1702a, 1702b, 1702c, 1702d are configured to contact and be contoured to align with the shape of the head of the subject. In some arrangements, the head pieces 1702a, 1702b, 1702c, 1702d are configured to be positioned at the four quadrants (or corners) of the head. In some arrangements, the head pieces 1702a, 1702b, 1702c, 1702d include padding therein to contact the head, as described herein.

In some arrangements, the connectors 1704a, 1704b, 1704c, 1704d located between the head pieces 1702a, 1702b, 1702c, 1702d provide an inward force between the head pieces 1702a, 1702b, 1702c, 1702d such that the head pieces 1702a, 1702b, 1702c, 1702d are drawn into one another. For example, the connectors 1704a, 1704b, 1704c, 1704d can include springs therein to exert the inward force between the head pieces 1702a, 1702b, 1702c, 1702d (e.g., a spring tension force), and the springs can be calibrated to adjust the amount of spring force. In some arrangements, the head pieces 1702a, 1702b, 1702c, 1702d are manually pulled apart from one another such that the inward force provided by the connectors 1704a, 1704b, 1704c, 1704d allow the head pieces 1702a, 1702b, 1702c, 1702d to be held fast against the head when the headset 1700 is worn. In other arrangements, any suitable number of head pieces 1702a, 1702b, 1702c, 1702d and connectors 1704a, 1704b, 1704c, 1704d therebetween can be used to provide a stable headset.

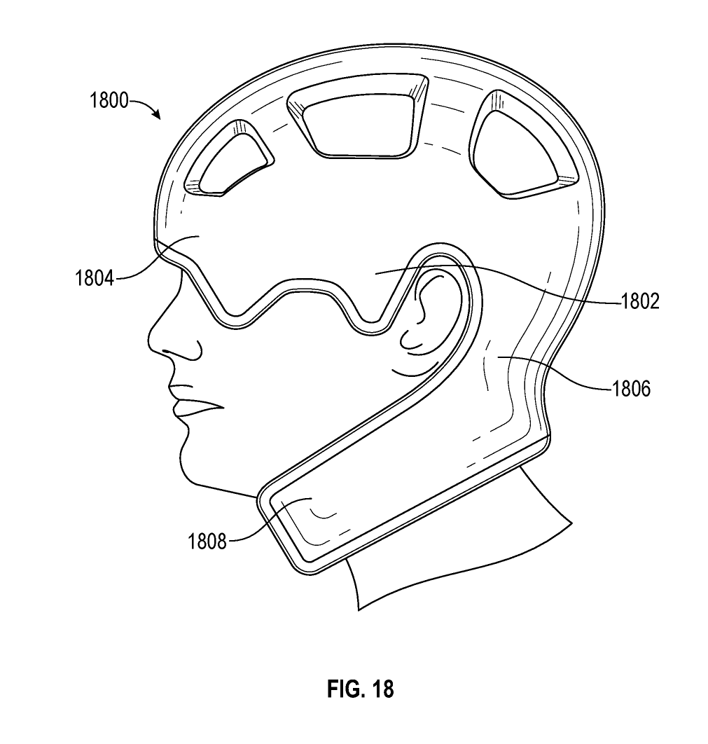

FIG. 18 illustrates a side view of a headset 1800 adapted to provide coverage with respect to multiple acoustic windows of a head according to various arrangements.

In some arrangements, the headset 1800 provides coverage with respect to multiple acoustic windows including, but not limited to, the temporal window 1802, the orbital window 1804, the occipital window 1806, and the mandibular window 1808. In some arrangements, the headset 1800 provides coverage (e.g., probe locations) over two or more acoustic windows. Each acoustic window enables acoustic energy to be provided to different arteries such that by providing access to one window, all relevant arteries may not be sufficiently covered. For example, some significant arteries covered by the temporal window 1802 include the middle cerebral artery (MCA), anterior cerebral artery (ACA), posterior cerebral artery (PCA), and terminal portion of the internal carotid artery (ICA). Some significant arteries covered by the orbital window 1804 include the ophthalmic artery and the ICA at the syphon level. Some significant arteries covered by the occipital window 1806 include the distal vertebral arteries and basilar artery. Some significant arteries covered by the mandibular window 1808 include the distal portions of the extracranial ICA. As such, in some arrangements, it is desirable to be able to apply acoustic energy to different arteries, and a headset that covers more than one window provides more coverage of different arteries. For example, to identify an unknown occlusion within the brain, measurements of different arteries are useful.

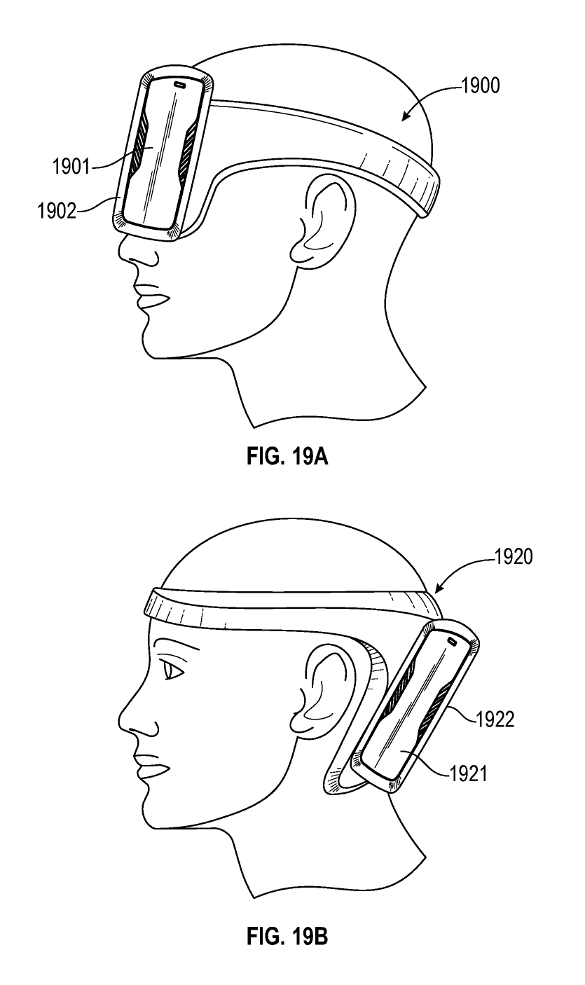

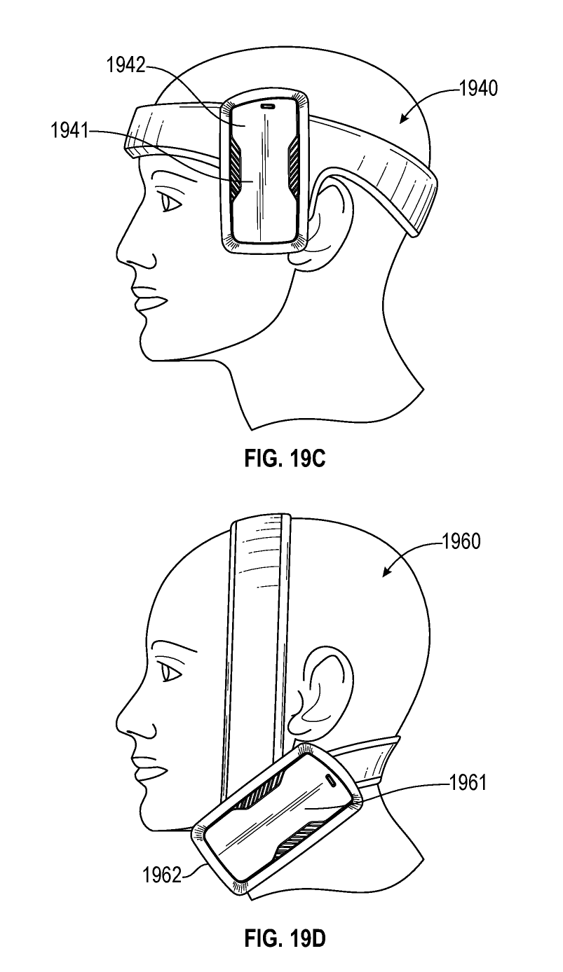

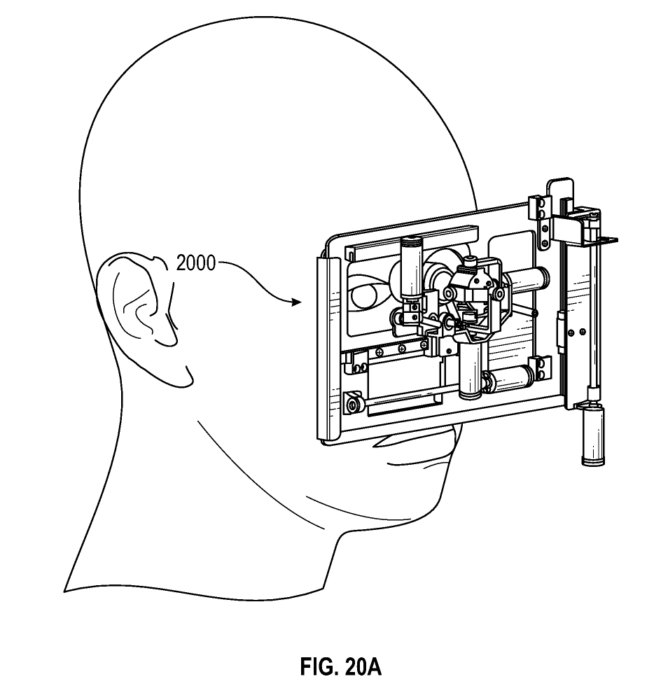

FIG. 19A, FIG. 19B, FIG. 19C, and FIG. 19D illustrate side views of headsets 1900, 1920, 1940, 1960 configured to cover different acoustic windows according to various arrangements.

In some arrangements, the different headsets 1900, 1920, 1940, 1960 provide coverage of different example ultrasound windows according. In some arrangements, the headsets 1900, 1920, 1940, 1960 are made from any suitable rigid material, such as, but not limited to, metal, hard plastic, aluminum, steel, titanium, magnesium, various alloys, rigid plastics, composites, carbon fiber, fiber glass, expanded foam, compression molded foam, SLA or FDM-made materials, RIM molding, ABS, TPO, nylon, PVC, fiber reinforced resins, or the like.

In some arrangements, the headset 1900 includes a device (e.g., probe unit) 1901 that is configured to be positioned next to and aligned with the orbital window 1902. In some arrangements, the headset 1920 includes a device (e.g., probe unit) 1921 that is configured to be positioned next to and aligned with the occipital window 1922. In some arrangements, the headset 1940 includes a device (e.g., probe unit) 1941 that is configured to be positioned next to and aligned with the temporal window 1942. In some arrangements, the headset 1960 includes a device (e.g., probe unit) 1961 that is configured to be positioned next to and aligned with the temporal window 1962. In some arrangements, the headsets 1900, 1920, 1940, 1960 include a plurality of probe units that are aligned with different respective acoustic windows.

In some arrangements, the probe units 1901, 1921, 1941, 1961 are ultrasound probes that can be used to perform ultrasound scans within the head, such as, but not limited to, transcranial Doppler (TCD), a transcranial color Doppler sonography (TCCS) with contrast-enhanced transcranial color Doppler sonography (CE-TCCS), or a robotic assembly that can move the probe independent of headset position. In some arrangements, the headsets 1900, 1920, 1940, 1960 provide any number of degrees of freedom for devices attached thereto. In various arrangements, the probe unit is a singular probe, an assembly, or an array of probes. In various arrangements, the probe unit is an electrical unit, such as, but not limited to, a phased array antenna system capable of beam steering.

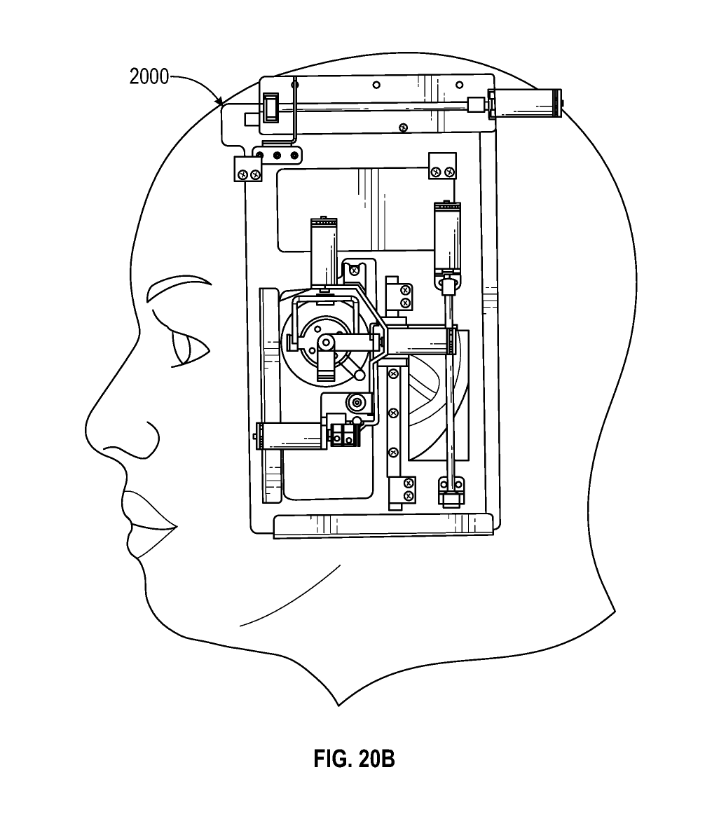

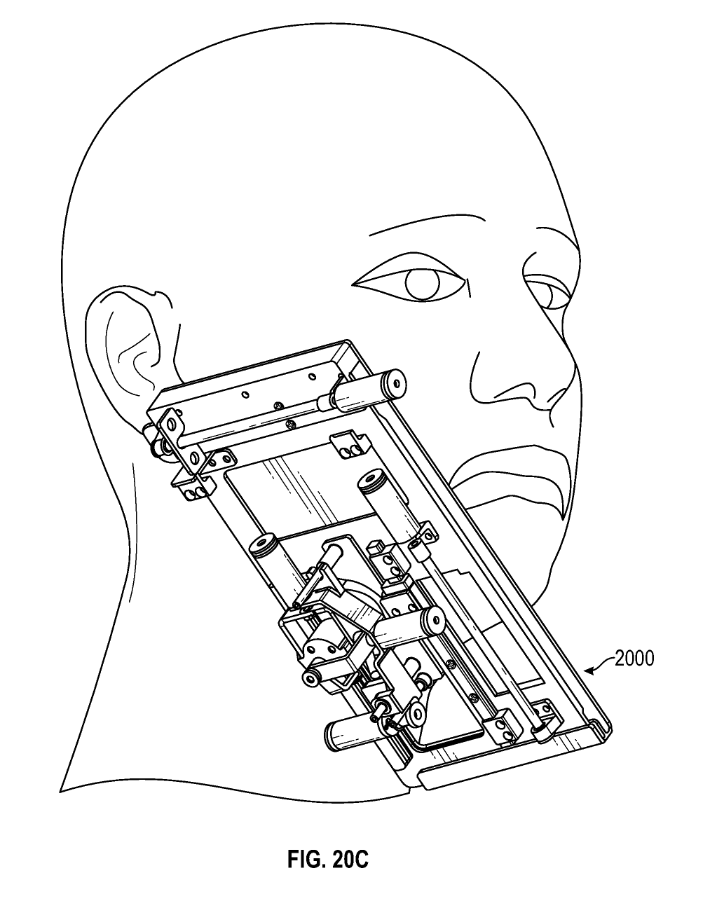

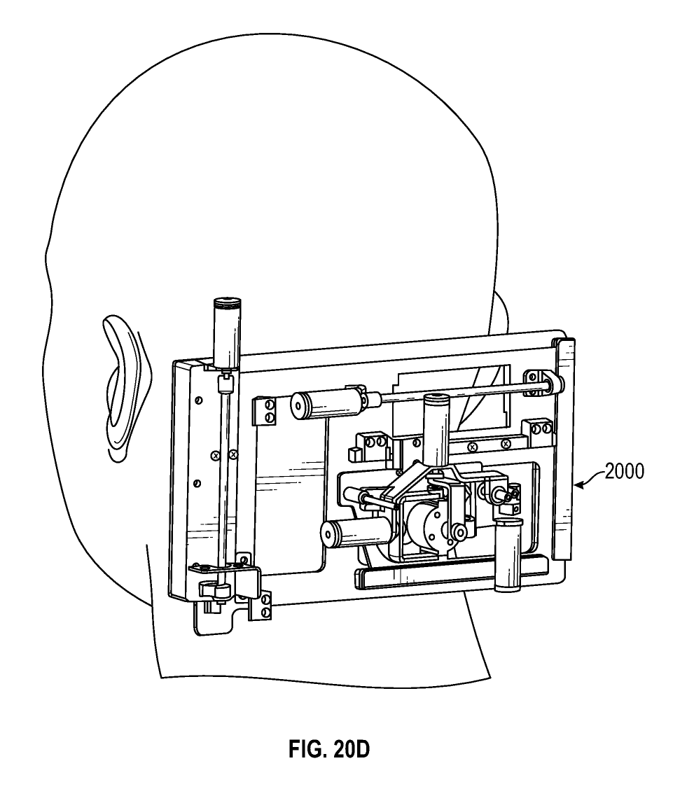

FIG. 20A, FIG. 20B, FIG. 20C, and FIG. 20D illustrate various views of a device 2000 positioned at different acoustic windows according to various arrangements.

FIGS. 20A-20D show desirable locations for a headset (e.g., those described herein) to hold a device (e.g., robotic mechanism including a probe attached thereto) 2002 with respect to anatomical acoustic windows. Accordingly, in some arrangements, the headsets described herein can be designed to allow access by a device to other acoustic windows of a subject, other than the temporal window. In further arrangements, the headsets described herein can be designed to allow access to multiple acoustic windows by multiple devices simultaneously. In various arrangements, features described with respect to a headset can be incorporated into other headsets, as is feasible (e.g., the quick release mechanism of headset 1600 can be incorporated into the headset 1100, and so on).

The above used terms, including “held fast,” “mount,” “attached,” “coupled,” “affixed,” “connected,” “secured,” and the like are used interchangeably. In addition, while certain arrangements have been described to include a first element as being “coupled” (or “attached,” “connected,” “fastened,” etc.) to a second element, the first element may be directly coupled to the second element or may be indirectly coupled to the second element via a third element.

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” Unless specifically stated otherwise, the term “some” refers to one or more. All structural and functional equivalents to the elements of the various aspects described throughout the previous description that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed as a means plus function unless the element is expressly recited using the phrase “means for.”

It is understood that the specific order or hierarchy of steps in the processes disclosed is an example of illustrative approaches. Based upon design preferences, it is understood that the specific order or hierarchy of steps in the processes may be rearranged while remaining within the scope of the previous description. The accompanying method claims present elements of the various steps in a sample order, and are not meant to be limited to the specific order or hierarchy presented.

The previous description of the disclosed implementations is provided to enable any person skilled in the art to make or use the disclosed subject matter. Various modifications to these implementations will be readily apparent to those skilled in the art, and the generic principles defined herein may be applied to other implementations without departing from the spirit or scope of the previous description. Thus, the previous description is not intended to be limited to the implementations shown herein but is to be accorded the widest scope consistent with the principles and novel features disclosed herein.

What is claimed is:

1. A headset configured to be placed on a head of a subject comprising:
a device comprising a probe; and
at least one adjustable mechanism configured to adjust a fit of the headset such that the device is aligned with an acoustic window of the head of the subject.

2. The headset of claim 1, wherein the at least one adjustable mechanism includes a temple adjustment mechanism configured to telescope to elongate or shorten a length of the headset at a temple of the head of the subject.

3. The headset of claim 2, wherein the at least one adjustable mechanism further includes a tip adjustment mechanism configured to telescope to elongate or shorten the length of the headset at an ear of the head of the subject.

4. The headset of claim 3, wherein the device is interposed between the temple adjustment mechanism and the tip adjustment mechanism along the length of the headset.

5. The headset of claim 1, wherein the at least one adjustable mechanism includes an adjustable pad located between the headset and the head of the subject and is configured to contact the head of the subject.

6. The headset of claim 5, wherein the adjustable pad has a first thickness and is replaceable with a replacement adjustable pad having a second thickness different from the first thickness.

7. The headset of claim 1, further comprising a head strap configured to span over a top of the head of the subject.

8. The headset of claim 1, wherein the device further includes a robotic mechanism attached to the probe for automatic control of the probe.

9. The headset of claim 1, wherein the probe includes an ultrasound probe.

10. The headset of claim 1, wherein the acoustic window includes a temporal acoustic window of the head of the subject.

11. A method of manufacturing a headset configured to be placed on a head of a subject comprising:
providing a device comprising a probe; and
providing at least one adjustable mechanism configured to adjust a fit of the headset such that the device is aligned with an acoustic window of the head of the subject.

12. The method of claim 11, wherein the at least one adjustable mechanism includes a temple adjustment mechanism configured to telescope to elongate or shorten a length of the headset at a temple of the head of the subject.

13. The method of claim 12, wherein the at least one adjustable mechanism further includes a tip adjustment mechanism configured to telescope to elongate or shorten the length of the headset at an ear of the head of the subject.

14. The method of claim 13, wherein the device is interposed between the temple adjustment mechanism and the tip adjustment mechanism along the length of the headset.

15. The method of claim 11, wherein the at least one adjustable mechanism includes an adjustable pad located between the headset and the head of the subject and is configured to contact the head of the subject.

16. The method of claim 15, wherein the adjustable pad has a first thickness and is replaceable with a replacement adjustable pad having a second thickness different from the first thickness.

17. The method of claim 11, further comprising providing a head strap configured to span over a top of the head of the subject.

18. The method of claim 11, wherein the device further includes a robotic mechanism attached to the probe for automatic control of the probe.

19. The method of claim 11, wherein the probe comprises an ultrasound probe.

20. The method of claim 11, wherein the acoustic window includes a temporal acoustic window of the head of the subject.
